(19) United States
(12) Patent Application Publication
(10) Pub. No.: US 2015/0359867 A1
(43) Pub. Date: Dec. 17, 2015

(54) OVARIAN CANCER VACCINES AND VACCINATION METHODS

(71) Applicants: IMMUNOCELLULAR THERAPEUTICS, LTD., Calabasas, CA (US); CEDARS-SINAI MEDICAL CENTER, Los Angeles, CA (US)

(72) Inventors: John S. Yu, Los Angeles, CA (US); Zhenhua Li, Rowland Heights, CA (US); Sandra Orsulic, Los Angeles, CA (US); Beth Y. Karlan, Beverly Hills, CA (US); James G. Bender, Rancho Santa Margarita, CA (US)

(21) Appl. No.: 14/766,711

(22) PCT Filed: Feb. 14, 2014
(86) PCT No.: PCT/US2014/016562

§ 371 Date: Aug. 7, 2015

Related U.S. Application Data
(60) Provisional application No. 61/764,801, filed on Feb. 14, 2013.

(51) Int. Cl. A61K 39/00; A61K 31/675; C12N 5/0784; A61K 45/06

(52) U.S. Cl. CPC A61K 39/0011; A61K 45/06; A61K 31/675; C12N 5/0639; A61K 2039/5154; A61K 2039/585; C12N 2506/115; C12N 2501/22; C12N 2501/2304; C12N 2501/25; C12N 2501/727; C12N 2501/998; C12N 2501/2313; C12N 2501/48; C12N 2501/599; C12N 2501/50; C12N 2501/11

(57) ABSTRACT

Compositions of multipeptide vaccines including tumor associated antigens, compositions of antigen presenting cell (e.g., dendritic cell) based vaccines presenting epitopes from tumor associated antigens, and methods of making same, are provided herein. Also, disclosed are methods for treating ovarian cancers using such vaccines.

### TECHNICAL FIELD

The disclosure relates generally to multivalent vaccine compositions, methods of making such compositions, and methods for the treatment of ovarian cancers.

### BACKGROUND

Epithelial ovarian cancer (EOC) is the most frequent cause of gynecologic cancer-related mortality in women (Jemal, A., et al., Global cancer statistics. CA Cancer J Clin, 2011, 61(2): p. 69-90). It was estimated that in 2008 (the most recent year numbers are available), approximately 21,204 women were diagnosed and 14,362 women died of disease in the US (see, www.cdc.gov/cancer/ovarian/statistics/index.htm). It is estimated that approximately 190,000 new cases will be diagnosed and 115,000 women will die from ovarian cancer per year world-wide. While advances in chemotherapy have been made over the past three decades, the overall 5 year survival for advanced stage disease remains less than 35%.

Initial response rates of advanced ovarian cancer to the standard upfront paclitaxel and carboplatin treatment approach is 75%, with complete clinical response rates near 55%. Unfortunately over 75% of subjects with complete clinical response are destined to relapse and succumb to their disease (Coukos, G. and S. C. Rubin, Chemotherapy resistance in ovarian cancer: new molecular perspectives. Obstet Gynecol, 1998, 91(5 Pt 1): p. 783-92). For most subjects, ovarian cancer will recur within two years, with median time to progression of 20-24 months for optimally surgically cytoreduced subjects and 12-18 months for subjects with suboptimal reduction. Response rates to second line chemotherapy are significantly lower, between 15-30%, depending on the length of progression free survival and the number of previous treatments. Once ovarian cancer has recurred, it is not considered curable and progression to death is usually inevitable, despite aggressive chemotherapy strategies. These facts elucidate the enormous unmet need for the development of alternate therapies in ovarian cancer (Coukos, G. and S. C. Rubin, Gene therapy for ovarian cancer. Oncology (Williston Park), 2001, 15(9): p. 1197-204, 1207; discussion 1207-8; Coukos, G., et al., Immunotherapy for gynaecological malignancies. Expert Opin Biol Ther, 2005, 5(9): p. 1193-210; Coukos, G., M. C. Courreges, and F. Benencia, Intraperitoneal oncolytic and tumor vaccination therapy with replication-competent recombinant virus: the herpes paradigm. Curr Gene Ther, 2003, 3(2): p. 113-25).

Immunotherapy is a form of cancer treatment that activates the immune system to attack and eradicate cancer cells. Cytotoxic T lymphocytes (“CTL”) are critical to a successful antitumor immune response. T cells that attack cancer cells require the presentation of tumor antigens to naïve T cells that undergo activation, clonal expansion, and ultimately exert their cytolytic effector function. Effective antigen presentation is essential to successful CTL effector function. Thus, the development of a successful strategy to initiate presentation of tumor antigens to T cells can be important to an immunotherapeutic strategy for cancer treatment.

With the clinical outcome of ovarian cancers being from poor to lethal, there exists a significant need for the development of novel therapeutic treatments.

### SUMMARY

This disclosure is based, at least in part, on the identification of antigens present on ovarian cancer stem cells. The identification of these antigens provides a method of targeting ovarian cancer stem cells within ovarian cancer by using a multivalent vaccine that stimulates T cells that recognize epitopes from these antigens thereby eliminating the cancer stem cell population within ovarian cancer. Targeting ovarian cancer stem cells can prevent recurrence of ovarian cancer. By using a multivalent vaccine comprising a combination of peptide epitopes, the methods described herein also provide a way of preventing or reducing the development of escape mutants.

Accordingly, compositions and methods for inducing immune responses in ovarian cancer patients against tumor antigens are provided herein. The compositions include multipeptide vaccines comprising HLA class I epitopes from at least five (e.g., 5, 6, 7 or 8) of the following antigens: mesothelin, HER-2/neu, IL-13 receptor α2, survivin, CD133, gp100, AIM-2, and epidermal growth factor receptor (EGFR). In certain embodiments, the at least five antigens are HER2, EGFR, IL13Rα2, survivin, and mesothelin. In certain embodiments, the at least five antigens are HER2, EGFR, IL13Rα2, survivin, and gp100. In certain embodiments, the at least five antigens are HER2, EGFR, IL13Rα2, survivin, and CD133. In certain embodiments, the at least five antigens are HER2, EGFR, IL13Rα2, survivin, and AIM2. In certain embodiments, the multipeptide vaccines described above further comprise an HLA class I epitope from RANBP2. The compositions also include antigen presenting cells (e.g., dendritic cells) that present epitopes comprising HLA class I epitopes from at least five of the above-listed eight tumor associated antigens. In certain embodiments, the at least five antigens are HER2, survivin, gp100, IL13Rα2, EGFR, and mesothelin. In certain embodiments, the at least five antigens are HER2, survivin, gp100, IL13Rα2, EGFR, and CD133. In certain embodiments, the at least five antigens are HER2, survivin, gp100, IL13Rα2, EGFR, and AIM2. In certain embodiments, the at least five antigens are HER2, survivin, gp100, IL13Rα2, EGFR, AIM2, and CD133. In certain embodiments, the at least five antigens are mesothelin, HER-2/neu, IL-13 receptor α2, survivin, CD133, gp100, AIM-2, and epidermal growth factor receptor (EGFR). In certain embodiments, the multipeptide vaccines described above further comprise an HLA class I epitope from RANBP2. It is believed that at least one epitope from each of five of the above-listed eight tumor associated antigens will give rise to an efficacious therapeutic. Accordingly, the methods described herein make use of such vaccines for the treatment of ovarian cancer.

In one aspect, the disclosure features a composition comprising at least one major histocompatibility complex (MHC) class I peptide epitope of at least five (5, 6, 7, or 8) antigens selected from the group consisting of mesothelin, HER-2/neu, IL-13 receptor α2, survivin, CD133, gp100, AIM-2, and epidermal growth factor receptor (EGFR). In certain embodiments, the at least five antigens are HER2, EGFR, IL13Rα2, survivin, and mesothelin. In certain embodiments, the at least five antigens are HER2, EGFR, IL13Rα2, survivin, and gp100. In certain embodiments, the at least five antigens are HER2, EGFR, IL13Rα2, survivin, and CD133. In certain embodiments, the at least five antigens are HER2, EGFR, IL13Rα2, survivin, and AIM2. In certain embodiments, the at least five antigens includes RANBP2. The epitopes of the at least five antigens may be stored individually or stored as a mixture of these epitopes. In some embodiments, the composition features at least one major histocompatibility complex (MHC) class I peptide epitope of at least six antigens, at least seven antigens, or eight antigens. In certain embodiments, the at least five antigens are HER2, survivin, gp100, IL13Rα2, EGFR, and mesothelin. In certain embodiments, the at least five antigens are HER2, survivin, gp100, IL13Rα2, EGFR, and CD133. In certain embodiments, the at least five antigens are HER2, survivin, gp100, IL13Rα2, EGFR, and AIM2. In certain embodiments, the at least five antigens are HER2, survivin, gp100, IL13Rα2, EGFR, AIM2, and CD133. In certain embodiments, the at least five antigens are mesothelin, HER-2/neu, IL-13 receptor α2, survivin, CD133, gp100, AIM-2, and epidermal growth factor receptor (EGFR). In certain embodiments, the at least five antigens further comprise an HLA class I epitope from RANBP2. In certain embodiments of this aspect, the MHC class I peptide epitope is an HLA-A2 epitope. In a specific embodiment, the MHC class I peptide epitope is an HLA-A0201 epitope. In certain embodiments, the peptides are synthetic. In some embodiments, the composition further comprises at least one MHC class II peptide epitope. In some embodiments, the composition further comprises an adjuvant. In some embodiments, the composition further comprises a pharmaceutically acceptable carrier.

In another aspect, the disclosure features a composition comprising isolated dendritic cells, wherein the dendritic cells present peptide sequences on their cell surface, wherein the peptide sequences comprise at least one major histocompatibility complex (MHC) class I peptide epitope of at least five antigens selected from the group consisting of mesothelin, HER-2/neu, IL-13 receptor α2, survivin, CD133, gp100, AIM-2, and epidermal growth factor receptor (EGFR). In certain embodiments, the at least five antigens are HER2, EGFR, IL13Rα2, survivin, and mesothelin. In certain embodiments, the at least five antigens are HER2, EGFR, IL13Rα2, survivin, and gp100. In certain embodiments, the at least five antigens are HER2, EGFR, IL13Rα2, survivin, and CD133. In certain embodiments, the at least five antigens are HER2, EGFR, IL13Rα2, survivin, and AIM2. In certain embodiments, the at least five antigens further comprise an HLA class I epitope from RANBP2. In some embodiments, the composition features at least one major histocompatibility complex (MHC) class I peptide epitope of at least six antigens, at least seven antigens, or eight antigens. In certain embodiments, the at least five antigens are HER2, survivin, gp100, IL13Rα2, EGFR, and mesothelin. In certain embodiments, the at least five antigens are HER2, survivin, gp100, IL13Rα2, EGFR, and CD133. In certain embodiments, the at least five antigens are HER2, survivin, gp100, IL13Rα2, EGFR, and AIM2. In certain embodiments, the at least five antigens are HER2, survivin, gp100, IL13Rα2, EGFR, AIM2, and CD 133. In certain embodiments, the at least five antigens are mesothelin, HER-2/neu, IL-13 receptor α2, survivin, CD133, gp100, AIM-2, and epidermal growth factor receptor (EGFR). In certain embodiments, the at least five antigens further comprise an HLA class I epitope from RANBP2. In certain embodiments of this aspect, the MHC class I peptide epitope is an HLA-A2 epitope. In a specific embodiment, the MHC class I peptide epitope is an HLA-A0201 epitope. In certain embodiments, the peptides are synthetic. In some embodiments, the composition further comprises at least one MHC class II peptide epitope. In some embodiments, the composition further comprises an adjuvant. In some embodiments, the composition further comprises a pharmaceutically acceptable carrier. In certain embodiments, the dendritic cells acquired the peptide epitopes in vitro by exposure to synthetic peptides comprising the peptide epitopes.

In another aspect, the disclosure features a method of treating an ovarian cancer, comprising administering to a subject in need thereof an effective amount of a composition described herein.

In yet another aspect, the disclosure features a method of killing ovarian cancer stem cells, comprising administering to a subject in need thereof an effective amount of a composition described herein.

In certain embodiments of the above two aspects, the methods further comprise administering a second agent prior to administering the subject with the composition, wherein the second agent is any agent that is useful in the treatment of ovarian cancer. Combination therapy may allow lower doses of multiple agents and/or modified dosing regimens, thus reducing the overall incidence of adverse effects. In some embodiments, the method further involves administering a chemotherapeutic agent prior to administering the subject with the composition. In certain embodiments, the subject is administered the chemotherapeutic agent half an hour to 3 days (e.g., 1 hour, 2 hours, 3 hours, 4 hours, 5 hours, 6 hours, 7 hours, 8 hours, 9 hours, 12 hours, 1 day, 1.5 days, 2 days, 2.5 days, 3 days) prior to administering the subject with the composition. In a specific embodiment, the chemotherapeutic agent is cyclophosphamide. In other embodiments, the chemotherapeutic agent is paclitaxel, altretamine, capecitabine, etoposide, gemcitabine, ifosfamide, irinotecan, doxorubicin, melphalan, pemetrexed, toptecan, or vinorelbine.

In yet another aspect, the disclosure features a process comprising the steps of: obtaining bone marrow derived mononuclear cells from a patient; culturing the mononuclear cells in vitro under conditions in which mononuclear cells become adherent to a culture vessel; selecting adherent mononuclear cells; culturing the adherent mononuclear cells in the presence of one or more cytokines under conditions in which the cells differentiate into antigen presenting cells; culturing the antigen presenting cells in the presence of peptides under conditions in which the antigen presenting cells present the peptides on major histocompatibility class I molecules. In certain embodiments, the peptides comprise amino acid sequences corresponding to at least one MHC class I peptide epitope of at least five, at least six, at least seven, or eight of the following antigens: mesothelin, HER-2/neu, IL-13 receptor α2, survivin, CD133, gp100, AIM-2, and EGFR. In some embodiments, the at least one MHC class I peptide epitope is a HLA-A2 epitope. In a specific embodiment, the HLA-A2 epitope is an HLA-A0201 epitope. In some embodiments, the one or more cytokines comprise granulocyte macrophage colony stimulating factor and interleukin-4 (IL-4). In some embodiments, the one or more cytokines comprise tumor necrosis factor-α (TNF-α). In certain embodiments, the bone marrow derived cells are obtained from a patient diagnosed with epithelial ovarian cancer.

“Epitope” means a short peptide derived from a protein antigen, wherein the peptide binds to a major histocompatibility complex (MHC) molecule and is recognized in the MHC-bound context by a T cell. The epitope may bind an MHC class I molecule (e.g., HLA-A1, HLA-A2 or HLA-A3) or an MHC class II molecule.

By “peptide” is meant not only molecules in which amino acid residues are joined by peptide (—CO—NH—) linkages, but also molecules in which the peptide bond is reversed. Such retro-inverso peptidomimetics may be made using methods known in the art, for example such as those described in Meziere et al., J. Immunol. 159, 3230-3237 (1997), incorporated herein by reference. This approach involves making pseudopeptides containing changes involving the backbone, and not the orientation of side chains. Retro-inverse peptides, which contain NH—CO bonds instead of CO—NH peptide bonds, are much more resistant to proteolysis. In addition, the term “peptide” also includes molecules where the peptide bond may be dispensed with altogether provided that an appropriate linker moiety which retains the spacing between the Cα atoms of the amino acid residues is used; it is particularly preferred if the linker moiety has substantially the same charge distribution and substantially the same planarity of a peptide bond.

“Treatment” and “treating,” as used herein refer to both therapeutic treatment and prophylactic or preventative measures, wherein the object is to inhibit or slow down (lessen) the targeted disorder (e.g., cancer, e.g., ovarian cancer) or symptom of the disorder, or to improve a symptom, even if the treatment is partial or ultimately unsuccessful. Those in need of treatment include those already diagnosed with the disorder as well as those prone or predisposed to contract the disorder or those in whom the disorder is to be prevented. For example, in tumor (e.g., cancer) treatment, a therapeutic agent can directly decrease the pathology of tumor cells, or render the tumor cells more susceptible to treatment by other therapeutic agents or by the subject's own immune system.

A “dendritic cell” or “DC” is an antigen presenting cell (APC) that typically expresses high levels of MHC molecules and co-stimulatory molecules, and lacks expression of (or has low expression of) markers specific for granulocytes, NK cells, B lymphocytes, and T lymphocytes, but can vary depending on the source of the dendritic cell. DCs are able to initiate antigen specific primary T lymphocyte responses in vitro and in vivo, and direct a strong mixed leukocyte reaction (MLR) compared to peripheral blood leukocytes, splenocytes, B cells and monocytes. Generally, DCs ingest antigen by phagocytosis or pinocytosis, degrade it, present fragments of the antigen at their surface and secrete cytokines.

By “ovarian cancer” is meant a cancerous growth arising from the ovaries. The term encompasses epithelial ovarian tumors, germ cell ovarian tumors, sex cord stromal ovarian tumors as well as metastatic cancers that spread to the ovaries.

Unless defined otherwise, technical and scientific terms used herein have the same meaning as commonly understood by one of ordinary skill in the art to which this disclosure belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); Sambrook and Russel, Molecular Cloning: A Laboratory Manual 3rd ed., Cold Spring Harbor Laboratory Press (Cold Spring Harbor, N.Y. 2001); and Lutz et al., Handbook of Dendritic Cells: Biology, Diseases and Therapies, J. Wiley & Sons (New York, N.Y. 2006), provide one skilled in the art with a general guide to many of the terms used in the present application. Although methods and materials similar or equivalent to those described herein can be used in the practice or testing of the present invention, suitable methods and materials are described below. All publications, patent applications, patents, and other references mentioned herein are incorporated herein by reference in their entirety. In case of conflict, the present specification, including definitions, will control. In addition, the materials, methods, and examples are illustrative only and not intended to be limiting.

Other features and advantages of the invention will be apparent from the following detailed description, the drawings, and from the claims.

### BRIEF DESCRIPTION OF THE DRAWINGS

FIG. 1A is a bar graph showing RNA expression of antigens in human ovarian cancer cell (1031AC) relative to human ovarian epithelial cell (HoEpic) based on quantitative real-time PCR analysis.

FIG. 1B is a bar graph showing RNA expression of antigens in cancer stem cell (1031CSC) relative to human ovarian epithelial cell (HoEpic) based on quantitative real-time PCR analysis.

FIG. 1C is a bar graph showing RNA expression of antigens in ovarian cancer daughter cells (1031ADC) relative to human ovarian epithelial cell (HoEpic) based on quantitative real-time PCR analysis.

FIG. 2A is a bar graph showing gene expression in human ovarian cancer stem cell (1031CSC) relative to human ovarian cancer cell (1031AC)

FIG. 2B is a bar graph showing gene expression in ovarian cancer daughter cell (1031ADC) relative to cancer stem cell (1031CSC).

FIG. 3 is a bar graph displaying the results of an IFN-γ ELISPOT assay of the antigen-specific T cell response to the T2 pulsed with CD133 HLA-A2 peptides of CD133p405, CD133p753, and CD133p804.

FIG. 4 is a bar graph showing the RNA expression of the indicated genes in a TCGA dataset (586 patient samples).

FIG. 5A is a graph depicting overall survival (OS) and RNA expression of the HER2 gene in human ovarian cancer (Dataset: TCGA, 557 human ovarian cancer patients).

FIG. 5B is a graph depicting overall survival (OS) and RNA expression of the MSLN gene in human ovarian cancer (Dataset: TCGA, 557 human ovarian cancer patients).

FIG. 5C is a graph depicting overall survival (OS) and RNA expression of the survivin gene in human ovarian cancer (Dataset: TCGA, 557 human ovarian cancer patients).

FIG. 5D is a graph depicting overall survival (OS) and RNA expression of the gp100 gene in human ovarian cancer (Dataset: TCGA, 557 human ovarian cancer patients).

FIG. 5E is a graph depicting overall survival (OS) and RNA expression of the EGFR gene in human ovarian cancer (Dataset: TCGA, 557 human ovarian cancer patients).

FIG. 5F is a graph depicting overall survival (OS) and RNA expression of the CD133gene in human ovarian cancer (Dataset: TCGA, 557 human ovarian cancer patients).

FIG. 5G is a graph depicting overall survival (OS) and RNA expression of the IL-13Rα2 gene in human ovarian cancer (Dataset: TCGA, 557 human ovarian cancer patients).

FIG. 6 is a graph showing overall survival (OS) and RNA expression of IL-13Rα2 in human ovarian cancer patients (Dataset GSE 9891, 285 human ovarian cancer patients).

### DETAILED DESCRIPTION

This disclosure relates in part to compositions that are useful to treat ovarian cancers. The compositions described herein include antigen presenting cells (e.g., dendritic cells) presenting peptide epitopes from five or more (e.g., 5, 6, 7, 8) tumor-associated antigens that are expressed on ovarian cancer stem cells or expressed at a higher levels on ovarian cancer stem cells than on differentiated ovarian tumor cells. Examples of such tumor-associated antigens include mesothelin, HER-2/neu, IL-13 receptor α2, survivin, CD133, gp100, AIM-2, and epidermal growth factor receptor (EGFR). The compositions described herein also include multipeptide mixtures of HLA epitopes of five or more of the above-listed tumor-associated antigens. Such multivalent vaccines are useful to prevent the development of escape mutants. Often, a tumor will evolve to turn off the expression of a particular tumor associated antigen, creating “escape mutants.” Thus, an immune response against multiple tumor antigens is more likely to provide effective therapy to deal with such mutants, and can provide significant therapeutic benefits for various patient populations. The multivalent vaccine compositions (multipeptide vaccines and APC vaccines) described herein are useful to raise a cytolytic T cell response against ovarian cancer stem cells thereby killing the cancer stem cells. The vaccines described can be used for the treatment of ovarian cancer and for the prevention or reduction of recurrence of ovarian cancer.

The compositions and methods of this disclosure feature at least five of the following antigens: mesothelin, HER-2/neu, IL-13 receptor α2, survivin, CD133, gp100, AIM-2, and epidermal growth factor receptor (EGFR). In one embodiment, the epitopes are MHC class I epitopes. In a specific embodiment, the epitopes are peptides that bind HLA-A2. The compositions and methods described herein may also feature one or more epitopes of other tumor associated antigens that are expressed on ovarian cancer stem cells; these epitopes may be MHC class I (e.g., HLA-A2) and/or class II epitopes.

This disclosure features the use of the peptides described herein (or polynucleotides encoding them) for active in vivo vaccination; for contacting autologous dendritic cells in vitro followed by introduction of the contacted dendritic cells in vivo to activate CTL responses; to activate autologous CTL in vitro followed by adoptive therapy (i.e., introducing the activated autologous CTL into a patient); and to activate CTL from healthy donors (MHC matched or mismatched) in vitro followed by adoptive therapy.

### Antigens

Mesothelin

Mesothelin is a differentiation antigen present on normal mesothelial cells and overexpressed in several human tumors, including mesothelioma, ovarian cancer, and pancreatic adenocarcinoma. The mesothelin gene encodes a precursor protein that is processed to yield the 40-kDa protein, mesothelin, which is attached to the cell membrane by a glycosylphosphatidyl inositol linkage and a 31-kDa shed fragment named megakaryocyte-potentiating factor. This protein is thought to play a role in cancer metastasis by mediating cell adhesion by binding to MUC16/CA-125.

Table 1 provides an amino acid sequence of the 622 amino acid human mesothelin protein (also available in GenBank under accession no. NP_{—}001170826.1). Exemplary sequences of mesothelin HLA epitopes are provided in Table 2.

HER-2

HER-2 (also known as HER-2/neu, and c-erbB2) is a 1255 amino acid transmembrane glycoprotein with tyrosine kinase activity. HER-2 is overexpressed in a variety of tumor types. This protein promotes tumor growth by activating a variety of cell signaling pathways including MAPK, PI3K/Akt, and PKC.

Table 1 provides an amino acid sequence of human HER-2 (also available in GenBank under accession no. NP_{—}004439.2). Exemplary sequences of HER-2 HLA are listed in Table 2.

IL-13 Receptor α2

IL-13 receptor α2 is a non-signaling component of the multimeric IL-13 receptor. Stimulation of this receptor activates production of TGF-β1, which inhibits cytotoxic T cell function. The human IL-13 receptor α2 amino acid sequence, which is 380 amino acids in length, is shown in Table 1 (also available in Genbank under accession no. NP_{—}000631.1). An exemplary sequence of an IL-13 receptor α2 HLA epitope is shown in Table 2.

Survivin

Survivin is a member of the inhibitor of apoptosis family. Survivin inhibits caspase activation, thereby leading to negative regulation of apoptosis or programmed cell death. Survivin is expressed highly in most human tumors and fetal tissue, but is completely absent in terminally differentiated cells. This fact makes survivin an ideal target for cancer therapy as cancer cells are targeted while normal cells are left alone.

Table 1 provides a sequence of human survivin which is 137 amino acids in length (also available in GenBank under accession no. NP_{—}001012270.1). Exemplary HLA epitopes of survivin are listed in Table 2.

CD133

The cell surface marker CD133 (Prominin 1) is expressed in several human cancers including brain cancer, colon cancer, hepatocellular carcinoma, prostate cancer, multiple myeloma, and melanoma. Table 1 provides an amino acid sequence of human CD133 (also available in GenBank under accession no. NP_{—}001139319.1). Exemplary HLA epitopes of survivin are listed in Table 2.

gp100

gp100 is a glycoprotein preferentially expressed in melanocytes. Table 1 provides an amino acid sequence of human gp100 (also available in GenBank under accession no. NP_{—}008859.1). Table 2 lists exemplary HLA epitopes from gp100.

AIM-2

AIM-2 is expressed in a variety of tumor types, including neuroectodermal tumors, and breast, ovarian and colon carcinomas. Table 1 provides an amino acid sequence of human AIM-2 (also available in GenBank under accession no. AAD51813.1). An exemplary sequence of an AIM-2 HLA epitope is shown in Table 2.

Epidermal Growth Factor Receptor (EGFR)

The epidermal growth factor receptor (EGFR; ErbB-1; HER1 in humans) is the cell-surface receptor for members of the epidermal growth factor family (EGF-family) of extracellular protein ligands. EGFR exists on the cell surface and is activated by binding of its specific ligands, including epidermal growth factor and transforming growth factor α (TGFα). Table 1 provides an amino acid sequence of human EGFR (also available in GenBank under accession no. NP_{—}005219.2). An exemplary sequence of an EGFR HLA epitope is listed in Table 2.

TABLE 1

| Tumor | |
|---|---|
| Antigen | Amino Acid Sequence |
| Mesothelin | MALPTARPLL GSCGTPALGS LLFLLFSLGW VQPSRTLAGE TGQEAAPLDG VLANPPNISS |
| | LSPRQLLGFP CAEVSGLSTE RVRELAVALA QKNVKLSTEQ LRCLAHRLSE PPEDLDALPL |
| | DLLLFLNPDA FSGPQACTRF FSRITKANVD LLPRGAPERQ RLLPAALACW GVRGSLLSEA |
| | DVRALGGLAC DLPGRFVAES AEVLLPRLVS CPGPLDQDQQ EAARAALQGG GPPYGPPSTW |
| | SVSTMDALRG LLPVLGQPII RSIPQGIVAA WRQRSSRDPS WRQPERTILR PRFRREVEKT |
| | ACPSGKKARE IDESLIFYKK WELEACVDAA LLATQMDRVN AIPFTYEQLD VLKHKLDELY |
| | PQGYPESVIQ HLGYLFLKMS PEDIRKWNVT SLETLKALLE VNKGHEMSPQ VATLIDRFVK |
| | GRGQLDKDTL DTLTAFYPGY LCSLSPEELS SVPPSSIWAV RPQDLDTCDP RQLDVLYPKA |
| | RLAFQNMNGS EYFVKIQSFL GGAPTEDLKA LSQQNVSMDL ATFMKLRTDA VLPLTVAEVQ |
| | KLLGPHVEGL KAEERHRPVR DWILRQRQDD LDTLGLGLQG GIPNGYLVLD LSMQEALSGT |
| | PCLLGPGPVL TVLALLLAST LA (SEQ ID NO: 1) |
| HER-2 | MELAALCRWG LLLALLPPGA ASTQVCTGTD MKLRLPASPE THLDMLRHLY QGCQVVQGNL |
| | ELTYLPTNAS LSFLQDIQEV QGYVLIAHNQ VRQVPLQRLR IVRGTQLFED NYALAVLDNG |
| | DPLNNTTPVT GASPGGLREL QLRSLTEILK GGVLIQRNPQ LCYQDTILWK DIFHKNNQLA |
| | LTLIDTNRSR ACHPCSPMCK GSRCWGESSE DCQSLTRTVC AGGCARCKGP LPTDCCHEQC |
| | AAGCTGPKHS DCLACLHFNH SGICELHCPA LVTYNTDTFE SMPNPEGRYT FGASCVTACP |
| | YNYLSTDVGS CTLVCPLHNQ EVTAEDGTQR CEKCSKPCAR VCYGLGMEHL REVRAVTSAN |
| | IQEFAGCKKI FGSLAFLPES FDGDPASNTA PLQPEQLQVF ETLEEITGYL YISAWPDSLP |
| | DLSVFQNLQV IRGRILHNGA YSLTLQGLGI SWLGLRSLRE LGSGLALIHH NTHLCFVHTV |
| | PWDQLFRNPH QALLHTANRP EDECVGEGLA CHQLCARGHC WGPGPTQCVN CSQFLRGQEC |
| | VEECRVLQGL PREYVNARHC LPCHPECQPQ NGSVTCFGPE ADQCVACAHY KDPPFCVARC |
| | PSGVKPDLSY MPIWKFPDEE GACQPCPINC THSCVDLDDK GCPAEQRASP LTSIISAVVG |
| | ILLVVVLGVV FGILIKRRQQ KIRKYTMRRL LQETELVEPL TPSGAMPNQA QMRILKETEL |
| | RKVKVLGSGA FGTVYKGIWI PDGENVKIPV AIKVLRENTS PKANKEILDE AYVMAGVGSP |
| | YVSRLLGICL TSTVQLVTQL MPYGCLLDHV RENRGRLGSQ DLLNWCMQIA KGMSYLEDVR |
| | LVHRDLAARN VLVKSPNHVK ITDFGLARLL DIDETEYHAD GGKVPIKWMA LESILRRRFT |
| | HQSDVWSYGV TVWELMTFGA KPYDGIPARE IPDLLEKGER LPQPPICTID VYMIMVKCWM |
| | IDSECRPRFR ELVSEFSRMA RDPQRFVVIQ NEDLGPASPL DSTFYRSLLE DDDMGDLVDA |
| | EEYLVPQQGF FCPDPAPGAG GMVHHRHRSS STRSGGGDLT LGLEPSEEEA PRSPLAPSEG |
| | AGSDVFDGDL GMGAAKGLQS LPTHDPSPLQ RYSEDPTVPL PSETDGYVAP LTCSPQPEYV |
| | NQPDVRPQPP SPREGPLPAA RPAGATLERP KTLSPGKNGV VKDVFAFGGA VENPEYLTPQ |
| | GGAAPQPHPP PAFSPAFDNL YYWDQDPPER GAPPSTFKGT PTAENPEYLG LDVPV |
| | (SEQ ID NO: 2) |
| IL-13 | MAFVCLAIGC LYTFLISTTF GCTSSSDTEI KVNPPQDFEI VDPGYLGYLY LQWQPPLSLD |
| receptor α2 | HFKECTVEYE LKYRNIGSET WKTIITKNLH YKDGFDLNKG IEAKIHTLLP WQCTNGSEVQ |
| | SSWAETTYWI SPQGIPETKV QDMDCVYYNW QYLLCSWKPG IGVLLDTNYN LFYWYEGLDH |
| | ALQCVDYIKA DGQNIGCRFP YLEASDYKDF YICVNGSSEN KPIRSSYFTF QLQNIVKPLP |
| | PVYLTFTRES SCEIKLKWSI PLGPIPARCF DYEIEIREDD TTLVTATVEN ETYTLKTTNE |
| | TRQLCFVVRS KVNIYCSDDG IWSEWSDKQC WEGEDLSKKT LLRFWLPFGF ILILVIFVTG |
| | LLLRKPNTYP KMIPEFFCDT (SEQ ID NO: 3) |
| Survivin | MGAPTLPPAW QPFLKDHRIS TFKNWPFLEG CACTPERMAE AGFIHCPTEN EPDLAQCFFC |
| | FKELEGWEPD DDPMQRKPTI RRKNLRKLRR KCAVPSSSWL PWIEASGRSC LVPEWLHHFQ |
| | GLFPGATSLP VGPLAMS (SEQ ID NO: 4) |
| CD133 | MALVLGSLLL LGLCGNSFSG GQPSSTDAPK AWNYELPATN YETQDSHKAG PIGILFELVH |
| | IFLYVVQPRD FPEDTLRKFL QKAYESKIDY DKIVYYEAGI ILCCVLGLLF IILMPLVGYF |
| | FCMCRCCNKC GGEMHQRQKE NGPFLRKCFA ISLLVICIII SIGIFYGFVA NHQVRTRIKR |
| | SRKLADSNFK DLRTLLNETP EQIKYILAQY NTTKDKAFTD LNSINSVLGG GILDRLRPNI |
| | IPVLDEIKSM ATAIKETKEA LENMNSTLKS LHQQSTQLSS SLTSVKTSLR SSLNDPLCLV |
| | HPSSETCNSI RLSLSQLNSN PELRQLPPVD AELDNVNNVL RTDLDGLVQQ GYQSLNDIPD |
| | RVQRQTTTVV AGIKRVLNSI GSDIDNVTQR LPIQDILSAF SVYVNNTESY IHRNLPTLEE |
| | YDSYWWLGGL VICSLLTLIV IFYYLGLLCG VCGYDRHATP TTRGCVSNTG GVFLMVGVGL |
| | SFLFCWILMI IVVLTFVFGA NVEKLICEPY TSKELFRVLD TPYLLNEDWE YYLSGKLFNK |
| | SKMKLTFEQV YSDCKKNRGT YGTLHLQNSF NISEHLNINE HTGSISSELE SLKVNLNIFL |
| | LGAAGRKNLQ DFAACGIDRM NYDSYLAQTG KSPAGVNLLS FAYDLEAKAN SLPPGNLRNS |
| | LKRDAQTIKT IHQQRVLPIE QSLSTLYQSV KILQRTGNGL LERVTRILAS LDFAQNFITN |
| | NTSSVIIEET KKYGRTIIGY FEHYLQWIEF SISEKVASCK PVATALDTAV DVFLCSYIID |
| | PLNLFWFGIG KATVFLLPAL IFAVKLAKYY RRMDSEDVYD DVETIPMKNM ENGNNGYHKD |
| | HVYGIHNPVM TSPSQH (SEQ ID NO: 5) |
| gp100 | MDLVLKRCLL HLAVIGALLA VGATKVPRNQ DWLGVSRQLR TKAWNRQLYP EWTEAQRLDC |
| | WRGGQVSLKV SNDGPTLIGA NASFSIALNF PGSQKVLPDG QVIWVNNTII NGSQVWGGQP |
| | VYPQETDDAC IFPDGGPCPS GSWSQKRSFV YVWKTWGQYW QVLGGPVSGL SIGTGRAMLG |
| | THTMEVTVYH RRGSRSYVPL AHSSSAFTIT DQVPFSVSVS QLRALDGGNK HFLRNQPLTF |
| | ALQLHDPSGY LAEADLSYTW DFGDSSGTLI SRALVVTHTY LEPGPVTAQV VLQAAIPLTS |
| | CGSSPVPGTT DGHRPTAEAP NTTAGQVPTT EVVGTTPGQA PTAEPSGTTS VQVPTTEVIS |
| | TAPVQMPTAE STGMTPEKVP VSEVMGTTLA EMSTPEATGM TPAEVSIVVL SGTTAAQVTT |
| | TEWVETTARE LPIPEPEGPD ASSIMSTESI TGSLGPLLDG TATLRLVKRQ VPLDCVLYRY |
| | GSFSVTLDIV QGIESAEILQ AVPSGEGDAF ELTVSCQGGL PKEACMEISS PGCQPPAQRL |
| | CQPVLPSPAC QLVLHQILKG GSGTYCLNVS LADTNSLAVV STQLIMPGQE AGLGQVPLIV |
| | GILLVLMAVV LASLIYRRRL MKQDFSVPQL PHSSSHWLRL PRIFCSCPIG ENSPLLSGQQ |
| | V (SEQ ID NO: 6) |
| AIM-2 | MVVLGMQTEE GHCIMLRGLA PSLGGTQVIC KVVGLPSSIG FNTSSHLLFP ATLQGAPTHF |
| | PCRWRQGGST DNPPA (SEQ ID NO: 7) |
| EGFR | MRPSGTAGAA LLALLAALCP ASRALEEKKV CQGTSNKLTQ LGTFEDHFLS LQRMFNNCEV |
| | VLGNLEITYV QRNYDLSFLK TIQEVAGYVL IALNTVERIP LENLQIIRGN MYYENSYALA |
| | VLSNYDANKT GLKELPMRNL QEILHGAVRF SNNPALCNVE SIQWRDIVSS DFLSNMSMDF |
| | QNHLGSCQKC DPSCPNGSCW GAGEENCQKL TKIICAQQCS GRCRGKSPSD CCHNQCAAGC |
| | TGPRESDCLV CRKFRDEATC KDTCPPLMLY NPTTYQMDVN PEGKYSFGAT CVKKCPRNYV |
| | VTDHGSCVRA CGADSYEMEE DGVRKCKKCE GPCRKVCNGI GIGEFKDSLS INATNIKHFK |
| | NCTSISGDLH ILPVAFRGDS FTHTPPLDPQ ELDILKTVKE ITGFLLIQAW PENRTDLHAF |
| | ENLEIIRGRT KQHGQFSLAV VSLNITSLGL RSLKEISDGD VIISGNKNLC YANTINWKKL |
| | FGTSGQKTKI ISNRGENSCK ATGQVCHALC SPEGCWGPEP RDCVSCRNVS RGRECVDKCN |
| | LLEGEPREFV ENSECIQCHP ECLPQAMNIT CTGRGPDNCI QCAHYIDGPH CVKTCPAGVM |
| | GENNTLVWKY ADAGHVCHLC HPNCTYGCTG PGLEGCPTNG PKIPSIATGM VGALLLLLVV |
| | ALGIGLFMRR RHIVRKRTLR RLLQERELVE PLTPSGEAPN QALLRILKET EFKKIKVLGS |
| | GAFGTVYKGL WIPEGEKVKI PVAIKELREA TSPKANKEIL DEAYVMASVD NPHVCRLLGI |
| | CLTSTVQLIT QLMPFGCLLD YVREHKDNIG SQYLLNWCVQ IAKGMNYLED RRLVHRDLAA |
| | RNVLVKTPQH VKITDFGLAK LLGAEEKEYH AEGGKVPIKW MALESILHRI YTHQSDVWSY |
| | GVTVWELMTF GSKPYDGIPA SEISSILEKG ERLPQPPICT IDVYMIMVKC WMIDADSRPK |
| | FRELIIEFSK MARDPQRYLV IQGDERMHLP SPTDSNFYRA LMDEEDMDDV VDADEYLIPQ |
| | QGFFSSPSTS RTPLLSSLSA TSNNSTVACI DRNGLQSCPI KEDSFLQRYS SDPTGALTED |
| | SIDDTFLPVP EYINQSVPKR PAGSVQNPVY HNQPLNPAPS RDPHYQDPHS TAVGNPEYLN |
| | TVQPTCVNST FDSPAHWAQK GSHQISLDNP DYQQDFFPKE AKPNGIFKGS TAENAEYLRV |
| | APQSSEFIGA (SEQ ID NO: 8) |

TABLE 2
Tumor Antigen Peptide Epitopes

| Tumor | Position in | |
|---|---|---|
| Antigen | Sequence | Peptide Sequence |
| Mesothelin | 20-28 | SLLFLLFSL (SEQ ID NO: 9) |
| Mesothelin | 23-31 | FLLFSLGWV (SEQ ID NO: 10) |
| Mesothelin | 530-538 | VLPLTVAEV (SEQ ID NO: 11) |
| Mesothelin | 547-556 | KLLGPHVEGL (SEQ ID NO: 12) |
| | (wt) | |
| Mesothelin | 547-556 | KLLGPHVLGL (SEQ ID NO: 13) |
| | (554L) | |
| Mesothelin | 547-556 | KLLGPHVLGV (SEQ ID NO: 14) |
| | (554L/556V)) | |
| Mesothelin | 547-556 | KMLGPHVLGV (SEQ ID NO: 15) |
| | (548M/554L/556V | |
| Mesothelin | 547-556 | KMLGPHVLGL (SEQ ID NO: 16) |
| | (548M/554L) | |
| Mesothelin | 547-556 | KILGPHVLGL (SEQ ID NO: 17) |
| | (5481/5540 | |
| Mesothelin | 547-556 | YLLGPHVLGV (SEQ ID NO: 18) |
| | (547Y/554L/556V) | |
| Mesothelin | 547-556 | YLLGPHVLGL (SEQ ID NO: 19) |
| | (547Y/554L) | |
| HER-2 | 5-13 | ALCRWGLLL (SEQ ID NO: 20) |
| HER-2 | 8-16 | RWGLLLALL (SEQ ID NO: 21) |
| HER-2 | 63-71 | TYLPTNASL (SEQ ID NO: 22) |
| HER-2 | 106-114 | QLFEDNYAL (SEQ ID NO: 23) |
| HER-2 | 369-377 | KIFGSLAFL (SEQ ID NO: 24) |
| HER-2 | 435-443 | ILHNGAYSL (SEQ ID NO: 25) |
| HER-2 | 654-662 | IISAVVGIL (SEQ ID NO: 26) |
| HER-2 | 665-673 | VVLGVVFGI (SEQ ID NO: 27) |
| HER-2 | 689-697 | RLLQETELV (SEQ ID NO: 28) |
| HER-2 | 754-762 | VLRENTSPK (SEQ ID NO: 29) |
| HER-2 | 773-782 | VMAGVGSPYV (SEQ ID NO: 30) |
| HER-2 | 780-788 | PYVSRLLGI (SEQ ID NO: 31) |
| HER-2 | 789-797 | CLTSTVQLV (SEQ ID NO: 32) |
| HER-2 | 799-807 | QLMPYGCLL (SEQ ID NO: 33) |
| HER-2 | 835-842 | YLEDVRLV (SEQ ID NO: 34) |
| HER-2 | 851-859 | VLVKSPNHV (SEQ ID NO: 35) |
| HER-2 | 883-899 | KVPIKWMALESILRRRF (SEQ ID NO: 36) |
| HER-2 | 952-961 | YMIMVKCWMI (SEQ ID NO: 37) |
| HER-2 | 971-979 | ELVSEFSRM (SEQ ID NO: 38) |
| IL-13 receptor | 345-354 | WLPFGFILI (SEQ ID NO: 39) |
| α2 | | |
| Survivin | 18-28 | RISTFKNWPFL (SEQ ID NO: 40) |
| Survivin | 53-67 | DLAQMFFCFKELEGW (SEQ ID NO: 41) |
| | M57 | |
| Survivin | 95-104 | ELTLGEFLKL (SEQ ID NO: 42) |
| Survivin | 96-104 | LTLGEFLKL (SEQ ID NO: 43) |
| | wt | |
| Survivin | 96-104 | LMLGEFLKL (SEQ ID NO: 44) |
| | M2 m | |
| CD133 | 117-126 | LLFIILMPLV (SEQ ID NO: 45) |
| CD133 | 301-309 | SLNDPLCLV (SEQ ID NO: 46) |
| CD133 | 405-413 | ILSAFSVYV (SEQ ID NO: 47) |
| CD133 | 708-716 | GLLERVTRI (SEQ ID NO: 48) |
| CD133 | 804-813 | FLLPALIFAV (SEQ ID NO: 49) |
| gp100 | 71-78 | SNDGPTLI (SEQ ID NO: 50) |
| gp100 | 154-162 | KTWGQYWQV (SEQ ID NO: 51) |
| gp100 | 209-217 | ITDQVPFSV (SEQ ID NO: 52) |
| gp100 | 280-288 | YLEPGPVTA (SEQ ID NO: 53) |
| gp100 | 613-622 | SLIYRRRLMK (SEQ ID NO: 54) |
| gp100 | 614-622 | LIYRRRLMK (SEQ ID NO: 55) |
| gp100 | 619-627 | RLMKQDFSV (SEQ ID NO: 56) |
| gp100 | 639-647 | RLPRIFCSC (SEQ ID NO: 57) |
| gp100 | 476-485 | VLYRYGSFSV (SEQ ID NO: 58) |
| AIM-2 | | RSDSGQQARY (SEQ ID NO: 59) |
| EGFR | 853-861 | IXDFGLAKL (SEQ ID NO: 60) |

As noted above, the epitopes listed in Table 2 are only exemplary. One of ordinary skill in the art would be able to identify other epitopes for these tumor associated antigens. In addition, the ordinary artisan would readily recognize that the epitopes listed in Table 2 can be modified by amino acid substitutions to alter HLA binding (e.g., to improve HLA binding). The epitopes may be modified at one, two, three, four, five, or six positions and tested for HLA binding activity. For instance, one or two of the amino acid residues are altered (for example by replacing them with the side chain of another naturally occurring amino acid residue or some other side chain) such that the peptide is still able to bind to an HLA molecule in substantially the same way as a peptide consisting of the given amino acid sequence.

For example, a peptide may be modified so that it at least maintains, if not improves, the ability to interact with and bind a suitable MHC molecule, such as HLA-A0201, and so that it at least maintains, if not improves, the ability to generate activated CTL which can recognize and kill ovarian cancer cells. Positions 2 and 9 of an HLA-A2-binding nonamer are typically anchor residues. Modifications of these and other residues involved in binding HLA-A2 may enhance binding without altering CTL recognition (Tourdot et al., J. Immunol., 159:2391-2398 (1997)). Based on routine binding assays, those with the desired binding activity and those capable of inducing suitable T cell responsiveness can be selected for use.

The antigenic peptides described herein can be used in multipeptide vaccines or for loading antigen presenting cells which can then be used for vaccination. These epitopes stimulate a T cell mediated immune response (e.g., a cytotoxic T cell response) by presentation to T cells on MHC molecules. Therefore, useful peptide epitopes of mesothelin, HER-2/neu, IL-13 receptor α2, survivin, CD133, gp100, AIM-2, and epidermal growth factor receptor (EGFR) include portions of their amino acid sequences that bind to MHC molecules and in that bound state are presented to T cells.

Humans have three different genetic loci that encode MHC class I molecules (designated human leukocyte antigens (HLA)): HLA-A, HLA-B, and HLA-C. HLA class I in humans, and equivalent systems in other animals, are genetically very complex. For example, there are at least 110 alleles of the HLA-B locus and at least 90 alleles of the HLA-A locus. Although any HLA class I (or equivalent) molecule is useful for purposes of this disclosure, it is preferred if the stimulator cell present epitopes in an HLA class I molecule which occurs at a reasonably high frequency in the human population. It is well known that the frequency of HLA class I alleles varies between different ethnic groupings such as Caucasian, African, and Chinese. For example, in Caucasian populations the HLA class I molecule is typically encoded by an HLA-A2 allele, an HLA-A1 allele, an HLA-A3 allele, or an HLA-B27 allele. HLA-A2 is particularly preferred. Combinations of HLA molecules may also be used. For example, a combination of HLA-A2 and HLA-A3 covers about 75% of the Caucasian population. Humans also have three different loci for MHC class II genes: HLA-DR, HLA-DQ, and HLA-DP. Peptides that bind to MHC class I molecules are generally 8-10 amino acids in length. Peptides that bind to MHC class II molecules are generally 13 amino acids or longer (e.g., 12-17 amino acids long).

T cell epitopes can be identified by a number of different methods. Naturally processed MHC epitopes can be identified by mass spectrophotometric analysis of peptides eluted from antigen-loaded APC (e.g., APC that have taken up antigen, or that have been engineered to produce the protein intracellularly). After incubation at 37° C., cells are lysed in detergent and the MHC protein is purified (e.g., by affinity chromatography). Treatment of the purified MHC with a suitable chemical medium (e.g., under acidic conditions, e.g., by boiling in 10% acetic acid, as described in Sanchez et al., Proc. Natl. Acad. Sci. USA, 94(9): 4626-4630, 1997) results in the elution of peptides from the MHC. This pool of peptides is separated and the profile compared with peptides from control APC treated in the same way. The peaks unique to the protein expressing/fed cells are analyzed (for example by mass spectrometry) and the peptide fragments identified. This protocol identifies peptides generated from a particular antigen by antigen processing, and provides a straightforward means of isolating these antigens.

Alternatively, T cell epitopes are identified by screening a synthetic library of peptides that overlap and span the length of the antigen in an in vitro assay. For example, peptides that are 9 amino acids in length and that overlap by 5 amino acids can be used. The peptides are tested in an antigen presentation system that includes antigen presenting cells and T cells. T cell activation in the presence of APCs presenting the peptide can be measured (e.g., by measuring T cell proliferation or cytokine production) and compared to controls, to determine whether a particular epitope is recognized by the T cells.

Another way to identify T cell epitopes is by algorithmic analysis of sequences that have predictive binding to HLA (see, e.g., www.immuneepitope.org) followed by binding studies and confirmation with in vitro induction of peptide specific CD8 T cells.

The T cell epitopes described herein can be modified to increase immunogenicity. One way of increasing immunogenicity is by the addition of dibasic amino acid residues (e.g., Arg-Arg, Arg-Lys, Lys-Arg, or Lys-Lys) to the N- and C-termini of peptides. Taking mesothelin as an example, modified T cell epitopes would be RRKLLGPHVEGL, KLLGPHVEGLRR, and KK KLLGPHVEGL, KLLGPHVEGLKK, KR KLLGPHVEGL, KLLGPHVEGLKR, RK KLLGPHVEGL, KLLGPHVEGLRK. Another way of increasing immunogenicity is by amino acid substitutions to either enhance Major Histocompatibility Complex (MHC) binding by modifying anchor residues (“fixed anchor epitopes”), or enhance binding to the T cell receptor (TCR) by modifying TCR interaction sites (“heteroclitic epitopes”) (see, e.g., Sette and Fikes, Current Opinion in Immunology, 2003,15:461-5470). In some embodiments, the epitopes described herein can be modified at one, two, three, four, five, or six positions. Even non-immunogenic or low affinity peptides can be made immunogenic by modifying their sequence to introduce a tyrosine in the first position (see, e.g., Tourdot et al., Eur. J Immunol., 2000, 30:3411-3421).

The peptides can also include internal mutations that render them “superantigens” or “superagonists” for T cell stimulation. Superantigen peptides can be generated by screening T cells with a positional scanning synthetic peptide combinatorial library (PS-CSL) as described in Pinilla et al., Biotechniques, 13(6):901-5, 1992; Borras et al., J. Immunol. Methods, 267(1):79-97, 2002; U.S. Publication No. 2004/0072246; and Lustgarten et al., J. Immun. 176:1796-1805, 2006. In some embodiments, a superagonist peptide is a peptide shown in Table 2, above, with one, two, three, or four amino acid substitutions which render the peptide a more potent immunogen.

Antigenic peptides can be obtained by chemical synthesis using a commercially available automated peptide synthesizer. Chemically synthesized peptides can be precipitated and further purified, for example by high performance liquid chromatography (HPLC). Alternatively, the peptides can be obtained by recombinant methods using host cell and vector expression systems. “Synthetic peptides” includes peptides obtained by chemical synthesis in vitro as well as peptides obtained by recombinant expression. When tumor antigen peptides are obtained synthetically, they can be incubated with antigen presenting cells in higher concentrations (e.g., higher concentrations than would be present in a tumor antigen cell lysates, which includes an abundance of peptides from non-immunogenic, normal cellular proteins). This permits higher levels of MHC-mediated presentation of the tumor antigen peptide of interest and induction of a more potent and specific immune response, and one less likely to cause undesirable autoimmune reactivity against healthy non-cancerous cells.

### Multipeptide Vaccines

In formulating a multipeptide vaccine it is not only important to identify and characterize tumor-associated antigens expressed on the ovarian cancer, but also the combinations of different epitopes from the tumor-associated antigens that increase the likelihood of a response to more than one epitope for the patient. To counter the tumor's ability to evade therapies directed against it, the present disclosure utilizes epitopes from a variety of antigens in the vaccine. Specifically, in one embodiment, combinations or mixtures of at least one HLA epitope from one, two, three, four, five, six, seven, or eight of the following tumor-associated antigens are particularly useful for immunotherapeutic treatments: mesothelin, HER-2/neu, IL-13 receptor α2, survivin, CD133, gp100, AIM-2, and epidermal growth factor receptor (EGFR). More than one epitope from the same antigen can be used in the multipeptide vaccine. For example, the vaccine may contain at least one, at least two, at least three, or at least four different epitopes from any of the eight tumor associated antigens listed above. In addition one or more epitopes from antigens other than the eight listed above can also be used. Furthermore, a class II epitope(s) may also be included.

To induce CTL killing of ovarian cancer cells, or to treat ovarian cancer, or prevent or reduce recurrence of ovarian cancer, the multipeptide vaccines comprise at least one HLA epitope from at least five (e.g., five, six, seven, or eight) of the following antigens: mesothelin, HER-2/neu, IL-13 receptor α2, survivin, CD133, gp100, AIM-2, and epidermal growth factor receptor (EGFR). In some embodiments, the HLA epitopes are HLA-A2 epitopes.

Ovarian cancer stem cells can also be targeted for destruction by using multipeptide vaccines that comprise at least one HLA epitope from at least five (e.g., five, six, seven, or eight) of the following antigens: mesothelin, HER-2/neu, IL-13 receptor α2, survivin, CD133, gp100, AIM-2, and epidermal growth factor receptor (EGFR). In some embodiments, the HLA epitopes are HLA-A2 epitopes. These vaccines can not only induce CTL killing of ovarian cancer stem cells but also cells of the differentiated ovarian tumors.

In some embodiments, the multipeptide vaccines described herein comprise a mixture of peptides that include one or more (e.g., one, two, three, four, five, six, seven, eight, nine, ten eleven, twelve) HLA epitopes from five or more (e.g., five, six, seven, or eight) of the antigens listed in Table 2.

In certain embodiments, the multipeptide vaccines described herein comprise a mixture of peptides that include one or more of the following HLA epitopes (e.g., one, two, three) from five or more of the following antigens (e.g., five, six, seven, or eight):

(SEQ ID NO: 12)
KLLGPHVEGL;
(SEQ ID NO: 14)
KLLGPHVLGV;
(SEQ ID NO: 9)
SLLFLLFSL;
(SEQ ID NO: 11)
VLPLTVAEV
from mesothelin;
(SEQ ID NO: 43)
LTLGEFLKL;
(SEQ ID NO: 44)
LMLGEFLKL;
(SEQ ID NO: 42)
ELTLGEFLKL;
(SEQ ID NO: 40)
RISTFKNWPFL;
(SEQ ID NO: 41)
DLAQMFFCFKELEGW
from survivin;
(SEQ ID NO: 30)
VMAGVGSPYV;
(SEQ ID NO: 24)
KIFGSLAFL;
(SEQ ID NO: 26)
IISAVVGIL;
(SEQ ID NO: 20)
ALCRWGLLL;
(SEQ ID NO: 25)
ILHNGAYSL;
(SEQ ID NO: 28)
RLLQETELV;
(SEQ ID NO: 27)
VVLGVVFGI;
(SEQ ID NO: 37)
YMIMVKCWMI;
(SEQ ID NO: 61)
HLYQGCQVV;
(SEQ ID NO: 62)
YLVPQQGFFC;
(SEQ ID NO: 63)
PLQPEQLQV;
(SEQ ID NO: 64)
TLEEITGYL;
(SEQ ID NO: 65)
ALIHHNTHL;
(SEQ ID NO: 66)
PLTSIISAV
from HER-2/neu;
(SEQ ID NO: 67)
IMDQVPFSV;
(SEQ ID NO: 51)
KTWGQYWQV;
(SEQ ID NO: 68)
AMLGTHTMEV;
(SEQ ID NO: 52)
ITDQVPFSV;
(SEQ ID NO: 53)
YLEPGPVTA;
(SEQ ID NO: 69)
LLDGTATLRL;
(SEQ ID NO: 58)
VLYRYGSFSV;
(SEQ ID NO: 70)
SLADTNSLAV;
(SEQ ID NO: 56)
RLMKQDFSV;
(SEQ ID NO: 71)
RLPRIFCSC
from gp100;
(SEQ ID NO: 59)
RSDSGQQARY
from AIM-2;
(SEQ ID NO: 39)
WLPFGFILI
from IL13Rα2;
(SEQ ID NO: 47)
ILSAFSVYV;
(SEQ ID NO: 72)
YLQWIEFSI
from CD133;
and
(SEQ ID NO: 60)
IXDFGLAKL
from EGFR (where X is any amino acid).

The multipeptide vaccines of the present disclosure can contain mixtures of epitopes from HLA-A2 restricted epitopes alone; HLA-A2 restricted epitopes in combination with at least one HLA-A1 or HLA-A3 restricted epitope; HLA-A2 restricted epitopes in combination with at least one HLA-DR, HLA-DQ, and/or HLA-DP restricted epitope; or HLA-A2 restricted epitopes in combination with at least one HLA-A1 or HLA-A3 restricted epitope and at least one HLA-DR, HLA-DQ, and/or HLA-DP restricted epitope. The MHC class I and MHC class II epitopes can be from the same antigen or different antigens.

The multipeptide mixture can be administered with adjuvants to render the composition more immunogenic. Adjuvants include, but are not limited to, Freund's adjuvant, GM-CSF, Montanide (e.g., Montanide IMS 1312, Montanide ISA 206, Montanide ISA 50V, and Montanide ISA-51), 1018 ISS, aluminum salts, Amplivax®, AS15, BCG, CP-870,893, CpG7909, CyaA, dSLIM, flagellin or TLR5 ligands derived from flagellin, FLT3 ligand, IC30, IC31, Imiquimod (ALDARA®), resiquimod, ImuFact IMP321, Interleukins such as IL-2, IL-4, IL-7, IL-12, IL-13, IL-15, IL-21, IL-23, Interferon-α or -β, or pegylated derivatives thereof, IS Patch, ISS, ISCOMATRIX, ISCOMs, JuvImmune, LipoVac, MALP2, MF59, monophosphoryl lipid A, water-in-oil and oil-in-water emulsions, OK-432, OM-174, OM-197-MP-EC, ONTAK, OspA, PepTel® vector system, poly(lactid co-glycolid) [PLG]-based and dextran microparticles, talactoferrin SRL172, virosomes and other virus-like particles, YF-17D, VEGF trap, R848, beta-glucan, Pam3Cys, Aquila's QS21 stimulon, mycobacterial extracts and synthetic bacterial cell wall mimics, Ribi's Detox, Quil, Superfos, cyclophosphamide, sunitinib, bevacizumab, celebrex, NCX-4016, sildenafil, tadalafil, vardenafil, sorafenib, temozolomide, temsirolimus, XL-999, CP-547632, pazopanib, VEGF Trap, ZD2171, AZD2171, and anti-CTLA4 antibodies. CpG immunostimulatory oligonucleotides can be used to enhance the effects of adjuvants in a vaccine setting. In one embodiment, the multipeptide vaccine is administered with Montanide ISA-51 and/or GM-CSF.

The multipeptide compositions of the present disclosure can be administered parenterally (e.g., subcutaneous, intradermal, intramuscular, intraperitoneal) or orally. The peptides and optionally other molecules (e.g., adjuvants) can be dissolved or suspended in a pharmaceutically acceptable carrier. In addition, the multipeptide compositions of the present disclosure can contain buffers and/or excipients. The peptides can also be administered together with immune stimulating substances, such as cytokines The peptides of the multipeptide vaccine can be administered at doses of between 1 mg and 500 mg of peptide. This disclosure also features polynucleotides encoding the peptides of the multivalent vaccine. As an alternative to administering a patient with multipeptide vaccines, polynucleotides encoding the desired HLA epitopes can also be administered to the patient in need of treatment for ovarian cancer.

The peptides for use in the vaccine can be synthesized, for example, by using the Fmoc-polyamide mode of solid-phase peptide synthesis which is disclosed by Lu et al (1981) J. Org. Chem. 46, 3433 and the references therein. The peptides described herein can be purified by any one, or a combination of, techniques such as recrystallization, size exclusion chromatography, ion-exchange chromatography, hydrophobic interaction chromatography, and reverse-phase high performance liquid chromatography using e.g. acetonitrile/water gradient separation. Analysis of peptides can be carried out using thin layer chromatography, electrophoresis, in particular capillary electrophoresis, solid phase extraction (CSPE), reverse-phase high performance liquid chromatography, amino-acid analysis after acid hydrolysis and by fast atom bombardment (FAB) mass spectrometric analysis, as well as MALDI and ESI-Q-TOF mass spectrometric analysis.

The peptides disclosed herein can have additional N- and/or C-terminally located stretches of amino acids that do not necessarily form part of the peptide that functions as the actual epitope for MHC molecules but can, nevertheless, be important for efficient introduction of the peptide into cells. The peptides described herein can also be modified to improve stability and/or binding to MHC molecules to elicit a stronger immune response. Methods for such an optimization of a peptide sequence are well known in the art and include, for example, the introduction of reverse peptide bonds or non-peptide bonds. Peptides comprising the sequences described herein can be synthesized with additional chemical groups present at their amino and/or carboxy termini, to enhance, for example, the stability, bioavailability, and/or affinity of the peptides. For example, hydrophobic groups such as carbobenzoxyl, dansyl, t-butyloxycarbonyl, acetyl, or a 9-fluorenylmethoxy-carbonyl group can be added to the peptides' amino terminus. Additionally, hydrophobic, t-butyloxycarbonyl, or amido groups can be added to the peptides' carboxy terminus. Further, all peptides described herein can be synthesized to alter their steric configuration. For example, the D-isomer of one or more of the amino acid residues of the peptides can be used, rather than the usual L-isomer. Still further, at least one of the amino acid residues of the peptides can be substituted by one of the well-known, non-naturally occurring amino acid residues. Alterations such as these can serve to increase the stability, bioavailability and/or binding action of the peptides of the disclosure. The peptides described herein can also be modified with polyethyleneglycol (PEG) and other polymers to extend their half-lives.

Once each peptide is prepared, it can be solubilized, sterile-filtered, and either stored by itself or mixed with the other peptides of the multipeptide vaccine and stored, at low temperatures (e.g., −80° C.) and protected from light.

### Preparation of Antigen Presenting Cells

Antigen-presenting cells (APCs) are cells that display antigens complexed with major histocompatibility complex (MHC) proteins on their surfaces. T cells cannot recognize, and therefore do not react to, “free” antigen. APCs process antigens and present them to T cells. T cells may recognize these complexes using their T-cell receptors (TCRs). Examples of APCs include dendritic cells, macrophages, B cells, and certain activated epithelial cells. Dendritic cells (DCs) include myeloid dendritic cells and plasmacytoid dendritic cells. APCs, suitable for administration to subjects (e.g., cancer patients), can be isolated or obtained from any tissue in which such cells are found, or can be otherwise cultured and provided.

APCs (e.g., DCs) can be found, by way of example, in the bone marrow or PBMCs of a mammal, in the spleen of a mammal, or in the skin of a mammal (i.e., Langerhans cells, which possess certain qualities similar to that of DC, may be found in the skin) For example, bone marrow can be harvested from a mammal and cultured in a medium that promotes the growth of DC. GM-CSF, IL-4 and/or other cytokines (e.g., TNF-α), growth factors and supplements can be included in this medium. After a suitable amount of time in culture in medium containing appropriate cytokines (e.g., suitable to expand and differentiate the DCs into mature DCs, e.g., 4, 6, 8, 10, 12, or 14 days), clusters of DC are cultured in the presence of epitopes of antigens of interest (e.g., in the presence of a mixture of at least one epitope from at least five, six, seven, or eight, of the following antigens: mesothelin, HER-2/neu, IL-13 receptor α2, survivin, CD133, gp100, AIM-2, and epidermal growth factor receptor (EGFR)) and harvested for use in a cancer vaccine using standard techniques.

Examples of epitopes that can be used for culturing with the APCs are listed in Table 2. In some embodiments, the APCs (e.g., DCs) are cultured with a mixture of peptides that include one or more (e.g., one, two, three, four, five, six, seven, eight, nine, ten, eleven, twelve) of HLA epitopes from five or more (e.g., five, six, seven, or eight) of the antigens listed in Table 2.

In certain embodiments, the APCs (e.g., DCs) are cultured with a mixture of peptides that include one or more of the following HLA epitopes (e.g., one, two, or three) from five or more of the following antigens (e.g., five, six, seven, or eight):

(SEQ ID NO: 12)
KLLGPHVEGL;
(SEQ ID NO: 14)
KLLGPHVLGV;
(SEQ ID NO: 9)
SLLFLLFSL;
(SEQ ID NO: 11)
VLPLTVAEV
from mesothelin;
(SEQ ID NO: 43)
LTLGEFLKL;
(SEQ ID NO: 44)
LMLGEFLKL;
(SEQ ID NO: 42)
ELTLGEFLKL;
(SEQ ID NO: 40)
RISTFKNWPFL;
(SEQ ID NO: 41)
DLAQMFFCFKELEGW
from survivin;
(SEQ ID NO: 30)
VMAGVGSPYV;
(SEQ ID NO: 24)
KIFGSLAFL;
(SEQ ID NO: 26)
IISAVVGIL;
(SEQ ID NO: 20)
ALCRWGLLL;
(SEQ ID NO: 25)
ILHNGAYSL;
(SEQ ID NO: 28)
RLLQETELV;
(SEQ ID NO: 27)
VVLGVVFGI;
(SEQ ID NO: 37)
YMIMVKCWMI;
(SEQ ID NO: 61)
HLYQGCQVV;
(SEQ ID NO: 62)
YLVPQQGFFC;
(SEQ ID NO: 63)
PLQPEQLQV;
(SEQ ID NO: 64)
TLEEITGYL;
(SEQ ID NO: 65)
ALIHHNTHL;
(SEQ ID NO: 66)
PLTSIISAV
from HER-2/neu;
(SEQ ID NO: 67)
IMDQVPFSV;
(SEQ ID NO: 51)
KTWGQYWQV;
(SEQ ID NO: 68)
AMLGTHTMEV;
(SEQ ID NO: 52)
ITDQVPFSV;
(SEQ ID NO: 53)
YLEPGPVTA;
(SEQ ID NO: 69)
LLDGTATLRL;
(SEQ ID NO: 58)
VLYRYGSFSV;
(SEQ ID NO: 70)
SLADTNSLAV;
(SEQ ID NO: 56)
RLMKQDFSV;
(SEQ ID NO: 71)
RLPRIFCSC
from gp100;
(SEQ ID NO: 59)
RSDSGQQARY
from AIM-2;
(SEQ ID NO: 39)
WLPFGFILI
from IL13Ra2;
(SEQ ID NO: 47)
ILSAFSVYV;
(SEQ ID NO: 72)
YLQWIEFSI
from CD133;
and
(SEQ ID NO: 60)
IXDFGLAKL
from EGFR (where X is any amino acid).

In certain embodiments, the epitopes are cultured with an APC (e.g., DC) are HLA-A2 epitopes. In addition to the HLA-A2 epitopes, the APCs can also be expanded in the presence of MHC class II epitopes and/or other HLA epitopes (e.g., HLA-A1 and/or HLA-A3). Epitopes of the antigens (e.g., isolated, purified peptides, or synthetic peptides) can be added to cultures at a concentration of 1 μg/ml-50 μg/ml per epitope, e.g., 2, 5, 10, 15, 20, 25, 30, or 40 μg/ml per epitope. Subject-specific APC vaccines (e.g., DC vaccines) are produced, carefully labeled, and stored. Single doses of the peptide-loaded (e.g., 1 to 50×10⁶ cells) APCs (e.g., DCs) can be cryopreserved in human serum albumin containing 10% dimethyl sulphoxide (DMSO) or in any other suitable medium for future use.

In one exemplary method of preparing APC (e.g., DC), the APC are isolated from a subject (e.g., a human) according to the following procedure. Mononuclear cells are isolated from blood using leukapheresis (e.g., using a COBE Spectra Apheresis System). The mononuclear cells are allowed to become adherent by incubation in tissue culture flasks for 2 hours at 37° C. Nonadherent cells are removed by washing. Adherent cells are cultured in medium supplemented with granulocyte macrophage colony stimulating factor (GM-CSF) (800 units/ml, clinical grade, Immunex, Seattle, Wash.) and interleukin-4 (IL-4) (500 units/ml, R&D Systems, Minneapolis, Minn.) for five days. On day five, TNF-α is added to the culture medium for another 3-4 days. On day 8 or 9, cells are harvested and washed, and incubated with peptide antigens for 16-20 hours on a tissue rotator. Peptide antigens are added to the cultures at a concentration of about 10 μg/ml to about 20 μg/ml per epitope.

Various other methods can be used to isolate the APCs, as would be recognized by one of skill in the art. DCs occur in low numbers in all tissues in which they reside, making isolation and enrichment of DCs a requirement. Any of a number of procedures entailing repetitive density gradient separation, fluorescence activated cell sorting techniques, positive selection, negative selection, or a combination thereof, are routinely used to obtain enriched populations of isolated DCs. Guidance on such methods for isolating DCs can be found, for example, in O'Doherty et al., J. Exp. Med., 178: 1067-1078, 1993; Young and Steinman, J. Exp. Med., 171: 1315-1332, 1990; Freudenthal and Steinman, Proc. Nat. Acad. Sci. USA, 57: 7698-7702, 1990; Macatonia et al., Immunol., 67: 285-289, 1989; Markowicz and Engleman, J. Clin. Invest., 85: 955-961, 1990; Mehta-Damani et al., J. Immunol., 153: 996-1003, 1994; and Thomas et al., J. Immunol., 151: 6840-6852, 1993. One method for isolating DCs from human peripheral blood is described in U.S. Pat. No. 5,643,786.

The DCs prepared according to methods described herein present epitopes corresponding to the antigens at a higher average density than epitopes present on dendritic cells exposed to a tumor lysate (e.g., an ovarian cancer lysate). The relative density of one or more antigens on antigen presenting cells can be determined by both indirect and direct means. The primary immune response of naïve animals is roughly proportional to the antigen density of antigen presenting cells (Bullock et al., J. Immunol., 170:1822-1829, 2003). Relative antigen density between two populations of antigen presenting cells can therefore be estimated by immunizing an animal with each population, isolating B or T cells, and monitoring the specific immune response against the specific antigen by, e.g., tetramer assays, ELISPOT, or quantitative PCR.

Relative antigen density can also be measured directly. In one method, the antigen presenting cells are stained with an antibody that binds specifically to the MHC-antigen complex, and the cells are then analyzed to determine the relative amount of antibody binding to each cell (see, e.g., Gonzalez et al., Proc. Natl. Acad. Sci. USA, 102:4824-4829, 2005). Exemplary methods to analyze antibody binding include flow cytometry and fluorescence activated cell sorting. The results of the analysis can be reported e.g., as the proportion of cells that are positive for staining for an individual MHC-antigen complex or the average relative amount of staining per cell. In some embodiments, a histogram of relative amount of staining per cell can be created.

In some embodiments, antigen density can be measured directly by direct analysis of the peptides bound to MHC, e.g., by mass spectrometry (see, e.g., Purcell and Gorman, Mol. Cell. Proteomics, 3:193-208, 2004). Typically, MHC-bound peptides are isolated by one of several methods. In one method, cell lysates of antigen presenting cells are analyzed, often following ultrafiltration to enrich for small peptides (see, e.g., Falk et al., J. Exp. Med., 174:425-434, 1991; Rotzxhke et al., Nature, 348:252-254, 1990). In another method, MHC-bound peptides are isolated directly from the cell surface, e.g., by acid elution (see, e.g., Storkus et al., J. Immunother., 14:94-103, 1993; Storkus et al., J. Immunol., 151:3719-27, 1993). In another method, MHC-peptide complexes are immunoaffinity purified from antigen presenting cell lysates, and the MHC-bound peptides are then eluted by acid treatment (see, e.g., Falk et al., Nature, 351:290-296). Following isolation of MHC-bound peptides, the peptides are then analyzed by mass spectrometry, often following a separation step (e.g., liquid chromatography, capillary gel electrophoresis, or two-dimensional gel electrophoresis). The individual peptide antigens can be both identified and quantified using mass spectrometry to determine the relative average proportion of each antigen in a population of antigen presenting cells. In some methods, the relative amounts of a peptide in two populations of antigen presenting cells are compared using stable isotope labeling of one population, followed by mass spectrometry (see, e.g., Lemmel et al., Nat. Biotechnol., 22:450-454, 2004).

### Administration of Antigen Presenting Cell-Based Vaccine

The APC-based vaccine can be delivered to a patient (e.g., a patient having a gynecological cancer or a peritoneal cancer) or test animal by any suitable delivery route, which can include injection, infusion, inoculation, direct surgical delivery, or any combination thereof. In some embodiments, the cancer vaccine is administered to a human in the deltoid region or axillary region. For example, the vaccine is administered into the axillary region as an intradermal injection. In other embodiments, the vaccine is administered intravenously.

An appropriate carrier for administering the cells can be selected by one of skill in the art by routine techniques. For example, the pharmaceutical carrier can be a buffered saline solution, e.g., cell culture media, and can include DMSO for preserving cell viability.

In certain embodiments, the cells are administered in an infusible cryopreservation medium. The composition comprising the cells can include DMSO and hetastarch as cryoprotectants, Plasmalyte A and /or dextrose solutions and human serum albumin as a protein component.

The quantity of APC appropriate for administration to a patient as a cancer vaccine to effect the methods described herein and the most convenient route of such administration are based upon a variety of factors, as can the formulation of the vaccine itself. Some of these factors include the physical characteristics of the patient (e.g., age, weight, and sex), the physical characteristics of the tumor (e.g., location, size, rate of growth, and accessibility), and the extent to which other therapeutic methodologies (e.g., chemotherapy, and beam radiation therapy) are being implemented in connection with an overall treatment regimen. Notwithstanding the variety of factors one should consider in implementing the methods of the present disclosure to treat a disease condition, a mammal can be administered with from about 10⁵ to about 10⁸ APC (e.g., 10⁷ APC) in from about 0.05 mL to about 2 mL solution (e.g., saline) in a single administration. Additional administrations can be carried out, depending upon the above-described and other factors, such as the severity of tumor pathology. In one embodiment, from about one to about five administrations of about 10⁶ APC is performed at two-week intervals.

DC vaccination can be accompanied by other treatments. For example, a patient receiving DC vaccination can also be receiving chemotherapy, radiation, and/or surgical therapy before, concurrently, or after DC vaccination. Chemotherapy is used to shrink and slow cancer growth. Chemotherapy is recommended for most women having ovarian cancer after the initial surgery for cancer; however, sometimes chemotherapy is given to shrink the cancer before surgery. The number of cycles of chemotherapy treatment depends on the stage of the disease. Chemotherapy may neutralize antitumor immune response generated through vaccine therapy. In addition, chemotherapy can be combined safely with immunotherapy, with possibly additive or synergistic effects, as long as combinations are designed rationally. Examples of chemotherapeutic agents that can be used in treatments of patients with ovarian cancers include, but are not limited to, carboplatin, cisplatin, cyclophosphamide, docetaxel, doxorubicin, etoposide, gemcitabine, oxaliplatin, paclitaxel, taxol, topotecan, and vinorelbine. In one embodiment, a patient is treated with cyclophosphamide (intravenously 200 mg/kg) prior to APC (e.g., DC) vaccination. For example, a patient can be intravenously injected with cyclophosphasmide (200 mg/kg) one day before, or between 24 hours and one hour before, APC (e.g., DC) vaccination. Cyclophosphamide is an alkylating drug that is used for treating several types of cancer. Cyclophosphamide is an inactive pro-drug; it is converted and activated by the liver into two chemicals, acrolein and phosphoramide. Acrolein and phosphoramide are the active compounds, and they slow the growth of cancer cells by interfering with the actions of deoxyribonucleic acid (DNA) within the cancerous cells. Cyclophosphamide is, therefore, referred to as a cytotoxic drug. Methods of treating cancer using DC vaccination in conjunction with chemotherapy are described, e.g., in Wheeler et al., U.S. Pat. No. 7,939,090. In some embodiments, a patient receiving DC vaccination has already received chemotherapy, radiation, and/or surgical treatment for the gynecological or peritoneal cancer.

In addition to, or separate from chemotherapeutic treatment, a patient receiving DC vaccination can be treated with any other treatments that are beneficial for ovarian cancer. For example, a patient having ovarian cancer can be treated prior to, concurrently, or after DC vaccination with a COX-2 inhibitor, as described, e.g., in Yu and Akasaki, WO 2005/037995. In another embodiment, a patient receiving DC vaccination can be treated with bevacizumab (Avastin®) prior to, concurrently, or after DC vaccination.

### Immunological Testing

The antigen-specific cellular immune responses of vaccinated subjects can be monitored by a number of different assays, such as tetramer assays and ELISPOT. The following sections provide examples of protocols for detecting responses with these techniques. Additional methods and protocols are available. See e.g., Current Protocols in Immunology, Coligan, J. et al., Eds., (John Wiley & Sons, Inc.; New York, N.Y.).

### Tetramer Assay

Tetramers comprised of recombinant MHC molecules complexed with a peptide can be used to identify populations of antigen-specific T cells. To detect T cells specific for antigens such as HER-2, FBP and mesothelin, fluorochrome labeled specific peptide tetramer complexes (e.g., phycoerythrin (PE)-tHLA) containing peptides from these antigens can be synthesized and provided by Beckman Coulter (San Diego, Calif.). Specific CTL clone CD8 cells can be resuspended in a buffer, e.g., at 10⁵ cells/50 μl FACS buffer (phosphate buffer plus 1% inactivated FCS buffer). Cells can be incubated with 1 μl tHLA for a sufficient time, e.g., for 30 minutes at room temperature, and incubation can be continued for an additional time, e.g., 30 minutes at 4° C. with 10 μl anti-CD8 mAb (Becton Dickinson, San Jose, Calif.). Cells can be washed twice, e.g., in 2 ml cold FACS buffer, before analysis by FACS (Becton Dickinson).

### ELISPOT Assay

ELISPOT assays can be used to detect cytokine secreting cells, e.g., to determine whether cells in a vaccinated patient secrete cytokine in response to antigen, thereby demonstrating whether antigen-specific responses have been elicited. ELISPOT assay kits are supplied, e.g., from R & D Systems (Minneapolis, Minn.) and can be performed as described by the manufacturer's instructions.

Responder (R) 1×10⁵ patients' PBMC cells from before and after vaccination are plated in 96-well plates with nitrocellulose membrane inserts coated with capture Ab. Stimulator (S) cells (TAP-deficient T2 cells pulsed with antigen) are added at the R:S ratio of 1:1. After a 24-hour incubation, cells are removed by washing the plates 4 times. The detection Ab is added to each well. The plates are incubated at 4° C. overnight and the washing steps will be repeated. After a 2-hour incubation with streptavidin-AP, the plates are washed. Aliquots (100 μl) of BCIP/NBT chromogen are added to each well to develop the spots. The reaction is stopped, e.g., after 60 minutes, e.g., by washing with water. The spots can be scanned and counted with a computer-assisted image analysis (Cellular Technology Ltd, Cleveland, Ohio). When experimental values are significantly different from the mean number of spots against non-pulsed T2 cells (background values), as determined by a two-tailed Wilcoxon rank sum test, the background values can be subtracted from the experimental values.

In vitro Induction of CTL in Patient-Derived PBMCs

The following protocol can be used to produce antigen specific CTL in vitro from patient-derived PBMC. To generate dendritic cells, the plastic adherent cells from PBMCs can be cultured in AIM-V medium supplemented with recombinant human GM-CSF and recombinant human IL-4 at 37° C. in a humidified CO₂ (5%) incubator. Six days later, the immature dendritic cells in the cultures can be stimulated with recombinant human TNF-α for maturation. Mature dendritic cells can then be harvested on day 8, resuspended in PBS at 1×106 per mL with peptide (2 μg/mL), and incubated for 2 hours at 37° C. Autologous CD8+ T cells can be enriched from PBMCs using magnetic microbeads (Miltenyi Biotech, Auburn, Calif.). CD8+ T cells (2×10⁶ per well) can be co-cultured with 2×10⁵ per well peptide-pulsed dendritic cells in 2 mL/well of AIM-V medium supplemented with 5% human AB serum and 10 units/mL rhIL-7 (Cell Sciences) in each well of 24-well tissue culture plates. About 20 U/ml of IL-2 can be added 24 h later at regular intervals, 2 days after each restimulation.

On day 7, lymphocytes can be restimulated with autologous dendritic cells pulsed with peptide in AIM-V medium supplemented with 5% human AB serum, rhIL-2, and rhIL-7 (10 units/mL each). About 20 U/ml of IL-2 can be added 24 h later at regular intervals, 2 days after each restimulation. On the seventh day, after the three rounds of restimulation, cells can be harvested and tested the activity of CTL. The stimulated CD8+ cultured cells (CTL) can be co-cultured with T2 cells (a human TAP-deficient cell line) pulsed with 2 μg/ml Her-2, FBP, mesothelin or IL13 receptor α2 peptides. After 24 hours incubation, IFN-γ in the medium can be measured by ELISA assay.

### Pharmaceutical Compositions

In various embodiments, the present disclosure provides pharmaceutical compositions, e.g., including a pharmaceutically acceptable carrier along with a therapeutically effective amount of the vaccines described herein that include multipeptide vaccines and dendritic cells loaded with the antigens described herein. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier can be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it can come in contact, meaning that it must not carry a risk of toxicity, irritation, allergic response, immunogenicity, or any other complication that excessively outweighs its therapeutic benefits.

In various embodiments, the pharmaceutical compositions described herein can be formulated for delivery via any route of administration. “Route of administration” can refer to any administration pathway, whether or not presently known in the art, including, but not limited to, aerosol, nasal, transmucosal, transdermal, or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions can be in the form of solutions or suspensions for infusion or for injection, or as lyophilized powders.

The pharmaceutical compositions described herein can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 21st edition, Williams & Wilkins Pa., USA) (2005). In one embodiment, a therapeutically effective amount of the vaccine can comprise about 10⁶ to about 10⁸ tumor antigen-pulsed DC (e.g., 10⁶, 0.5×10⁷, 10⁷, 0.5×10⁸, 10⁸). In some embodiments, a therapeutically effective amount is an amount sufficient to reduce or halt tumor growth, and/or to increase survival of a patient.

### Kits

The present disclosure is also directed to kits to treat ovarian cancer. The kits are useful for practicing the inventive method of treating cancer with a vaccine comprising dendritic cells loaded with the antigens or multipeptide vaccines as described herein. The kit is an assemblage of materials or components, including at least one of the compositions described herein. Thus, in some embodiments, the kit includes a set of peptides for preparing cells for vaccination. The kit can also include agents for preparing cells (e.g., cytokines for inducing differentiation of DC in vitro). The disclosure also provides kits containing a composition including a vaccine comprising dendritic cells (e.g., cryopreserved dendritic cells) loaded with the antigens as described herein.

The exact nature of the components configured in the kits described herein depends on their intended purpose. For example, some embodiments are configured for the purpose of treating ovarian cancers. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Optionally, the kit also contains other useful components, such as, diluents, buffers, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in cancer treatments or in vaccinations. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing for example, a vaccine comprising dendritic cells loaded with epitopes from the antigens as described herein. The packaging material generally has an external label which indicates the contents and/or purpose of the kit and/or its components.

### EXAMPLES

### Example 1

### Identification of Tumor Associated Antigens Expressed on Ovarian Cancer Stem Cells

SKOV-3 and Ovarian Cancer Cells (882, 1078, 1082) Culture

Human ovarian cancer cell line SKOV-3 and 882, 1078, 1082 were cultured in McCoy's 5A medium (Mediatech, Herndon, Va.) supplied with 10% fetal bovine serum (Omega Scientific, Inc), Pen Strep Glutamine (100×) (Invitrogen). All cells were cultured in 5% CO₂ and in a 37° C. cell incubator (Forma Scientific, Inc).

### Human Ovarian Cancer Stem Cell Culture

Human ovarian cancers (882,1078 and 1082) cells were grown in Dulbecco's modified Eagle's medium DMEM/F12 medium (Invitrogen) containing 10% fetal bovine serum (FBS) as growth medium and plated at a density of 4×10⁶ cells per 75 cm² cell culture flask (Corning). The cells attached and grew as a monolayer in the flasks. These monolayer growing human ovarian cancer cells were switched into DMEM/F12 medium supplemented with B-27 (Invitrogen, Carlsbad, Calif.), 20 ng/ml of basic fibroblast growth factor, and 20 ng/ml of endothelial-derived growth factor (Peprotech, Rocky Hill, N.J.).

### Flow Cytometric Analysis

The human ovarian cancer cells (1×10⁶) were resuspended in 1% FBS-PBS stained with following specific antibodies: anti-HER2, anti-IL-13RA2, anti-CD184, anti-CD44, anti-Survivin, anti-CD133, anti-mesothelin, anti-CD24, anti-EGFR, anti-EphA2, anti-FLOR1, anti-nestin, anti-NY-ESO-1, anti-MAGE-A1, and anti-TRP-2. These antibodies were purchased from commercial sources as direct conjugates to either PE or FITC.

For intracellular antigens (gp100, AIM-2) staining, cells were permeabilized using Cytofix/Cytoperm kit (BD Biosciences) and stained with PE-conjugated 2nd antibody.

Flow cytometric analysis was performed using a CyAn™ flow cytometer (Beckman Coulter) and the data was analyzed using Summit (Dako, Carpinteria, Calif., USA) software.

CSC are a defined subset of tumor cells capable of self-renewal and give rise to the proliferating bulk of rapidly proliferating and differentiating cells in a tumor. CSC are responsible for recurrence in many cancers including ovarian cancer. CSC in ovarian cancer are isolated by culturing under non-differentiating non-adherent conditions where they form spheroids. These spheroids have been shown to occur in vivo and are related to metastases. Ovarian CSC from spheroid cultures have been characterized for their expression of stem cell related antigen.

Although others have characterized antigen expression on ovarian tumor cells, the antigens present on the CSC fraction of ovarian cancer cells have not been well characterized. Table 3 provides the results of experiments conducted to characterize antigens that are expressed or overexpressed on the CSC population from ovarian cancer.

TABLE 3
PerCent Positive Expression of Antigen

| | | 882 line | | 1078 line | | 1082line |
|---|---|---|---|---|---|---|
| Antigens | SKOV3 | Adherent | Spheroid | Adherent | Spheroid | Adherent |
| HER2 | 99.9 | 89.9 | 93.6 | 91.94 | 82.18 | 99.07 |
| IL-13R2 | 3.4 | 1.1 | 26.2 | 15.97 | 47.52 | 18.36 |
| CD184 | 0.6 | 10.8 | 18.4 | 11.22 | 40.21 | 17.67 |
| CD44 | 99.9 | 99.7 | 98.42 | 62.8 | 63.88 | 99.08 |
| Survivin | 6.7 | 1.9 | 21.4 | 11.9 | 67.17 | 23.19 |
| CD133 | 6.7 | 83.4 | 5.6 | 93.04 | 88.17 | 7.19 |
| Mesothelin | 7.4 | | | 1.11 | 12.41 | 3.31 |
| EGFR | 1.55 | | 8.05 | | 6.04 | 1.65 |
| CD24 | 31.32 | | 24.05 | | 78.78 | 39.75 |
| Nestin | 2.67 | | 51.25 | | 4.98 | 1.51 |
| GP100 | 14.07 | 3.8 | 10.2 | 23.59 | 0.2 | 7.8 |
| AIM-2 | 78.14 | 28.95 | 25.38 | 9.8 | 0.14 | 37.6 |
| TRP-2 | 0.1 | 0.34 | 1.12 | 1.43 | 0.1 | 0.37 |
| MAGE-A1 | 2.08 | 0.48 | 2.53 | 5.68 | 0.12 | 0.17 |
| NY-ESO-1 | 0.09 | 0.26 | 0.24 | 0.95 | 0.17 | 0.66 |

As shown in Table 3, the antigens expressed on differentiated tumor (adherent) include: HER2, IL-13Ra2 (subset), CD184 (subset), CD44, survivin (subset), CD133, gp100 (subset), AIM2 (subset). The antigens expressed, or with an increased proportion, on CSC tumor (spheroid) include: HER2, IL-13Ra2 (increased), CD184 (increased), CD44, survivin (increased), CD133, mesothelin (increased), CD24, gp100, AIM2 (subset), nestin, and EGFR.

MHC epitopes of these antigens can be used in a multivalent vaccine for treatment of ovarian cancer.

### Example 2

### Preparation of Autologous Dendritic Cells (DC)

Human leukocyte antigen A2 (HLA-A2 or A2) positive patients with ovarian cancer are identified. Peripheral blood mononuclear cells (PBMCs) are isolated from such patients between days −30 to −15 using leukapheresis. The COBE Spectra Apheresis System is used to harvest the mononuclear cell layer. Leukapheresis yields about 10¹⁰ peripheral blood mononuclear cells (PBMC). If these cells are not to be processed to prepare DCs shortly after they are harvested, the product is packaged in insulated led containers with temperature monitors to ensure that a temperature range of 2-18° C. is maintained.

For processing the PBMCs to prepare DCs, the PBMCs are allowed to become adherent for two hours at 37° C. in a tissue culture flask and washed in HBSS. PBMC are seeded at a density of 1.4×10⁶ cells/cm² in 185-cm² culture flasks (Nunc, Roskilde, Denmark) and allowed to adhere for 2 h at 37° C. Non-adherent cells are removed by washing four times. Adherent cells are cultured in RPMI 1640 supplemented with GM-CSF (Berlex) and IL-4 (R&D systems) for 5 days. On day 5, 50 ng/ml clinical grade TNF-α (R&D systems) is added to the culture medium for another 3-4 days. On days 8-9, DCs are harvested and washed three times. Ideally about 7×10⁹ DCs are needed for treatment.

### Example 3

### Preparation of Vaccines

Dendritic cells, prepared as described in Example 2, are washed three times in dPBS, resuspended at 5-10×10⁶ cells/ml in complete media and then co-incubated with tumor associated antigen peptides (20 μg/ml per antigen, reconstituted in 10% DMSO). The dendritic cells are incubated with the peptides at 37°/5% CO2 for 16-20 hours on a tissue rotator to facilitate interaction.

After production, each DC preparation is tested for viability and microbial growth, and undergoes additional quality testing prior to freezing. A certificate of analysis will be produced for each batch (one certificate of analysis for each patient). The DC preparation is then frozen as follows: DC are resuspended in cryo tubes at various concentrations (1×10⁷ cells per ml in autologous freezing medium (10% DMSO and 90% autologous serum), then immediately transferred to 2 ml cryo tubes (cryo tube vials, Nunc, Brand Products, Roskilde, Denmark), slowly frozen to −80° C. by using a cryo-freezing container (Nalgene cryo 1° C. freezing container, rate of cooling −1° C./min (Fisher Scientific, CA)) and finally transferred into the gas phase of liquid nitrogen until use.

The study treatments will be labeled in such a way to clearly identify the patient. It is imperative that only the patient's own (autologous) study treatment be administered to the same individual patient. For these reasons, the blood specimen is procured and handled according to a strict protocol to ensure optimal quality of the specimen and minimum transport time to and from the processing facility, as well as to ensure the unique identification of the specimen at all times including injection back into the patient.

### Example 4

### Analysis of Expression of Tumor Antigens in Human Ovarian Tumor Samples

Purpose: To determine if the antigens described in Example 1 are present on/in primary human ovarian cancer cells.

Materials & Methods: Patients were entered into an Institutional Review Board-approved protocol and signed an informed consent prior to tissue collection. For enzymatic digestion of solid tumors, tumor specimen was diced into RPMI-1640, washed and centrifuged at 800 rpm for 5 min at 15-22° C., resuspended in enzymatic digestion buffer (0.2 mg/ml collagenase and 30 units/ml DNase in RPMI-1640) before overnight rotation at room temperature. Cells were then washed and cryopreserved as single cell suspensions for later use. Some solid tumor samples were physically dissociated using a Bellco Cellector device. For antigen profiling, seven solid tumor samples were enzymatically digested overnight and two were physically dissociated. On the day of study, cells were thawed and stained with indicated antibodies for extracellular protein analysis or fixed and permeabilized for staining of intracellular antigens. Multiparameter phenotypic analysis was performed on gated viable tumor cells (EpCAM+, 7AAD negative, CD45 negative) using antibodies specific for the following eight proteins: mesothelin, HER2/neu, IL-13Rα2, survivin, AIM2, RANBP2, gp100, and CD133 and compared to staining achieved using isotype antibody. Antigen positive established tumor cell lines were used as positive control whenever possible. Acquisition was performed on a BD Canto II flow cytometer and analysis performed using Flo-Jo software.

The antibodies used for the flow cytometric immunofluorescence analysis were as follows: antibodies against human CD45, EpCAM, HER2 and IL-13Rα2 were purchased from Biolegend (San Diego, Calif.); antibodies against mesothelin and survivin were from R&D Systems (Minneapolis, Minn.); antibodies against AIM2, RANBP2 and gp100 were from Abcam (Cambridge, Mass.); and antibody against CD133 was from Miltenyi Biotec (Auburn, Calif.). 7-AAD viability staining solution was purchased from BD Bioscience.

The flow cytometric immunofluorescence analysis was performed as follows: cells were resuspended in FACS buffer consisting of PBS with 2% FBS (Gemini Bioproducts). 10⁶ cells in 100 μl were directly stained with fluoro-chrome-conjugated mAbs at 4° C. for 40 min in the dark. For unconjugated antibodies, second fluoro-chrome-conjugated antibodies were stained for another 20 minutes. For viability gating, cells were briefly stained with 7-AAD solution and analyzed for nonviable cell exclusion using a FACS Cantor II (BD Biosciences). Intracellular staining was according to eBiosciences protocol (San Diego, Calif.).

Results: In the study of nine primary human ovarian cancers, 38.6%±13.4% of all viable cells from solid tumor cell suspensions were EpCAM⁺ tumor cells, while 28.6%±15.3% were CD45⁺ leukocytes (Table 4). Leukocytes were comprised of CD14⁻ monocytes, T lymphocytes, and low numbers of B lymphocytes, as well as other (non-T, B, mono) cells not defined within the applied antibody cocktail.

TABLE 4
T. 1 Composition of cells from primary solid ovarian Note: This table contains samples prepared
by enzyme digestion of solid tumor only except as noted (*)

| | | % viable of total | | | % of viable cells | | | % of viable leukocytes | | | |
|---|---|---|---|---|---|---|---|---|---|---|---|
| Sample | Date collected | total | tumor | leuco | CD45+ | EpCam+ | CD45− | T cells | B cells | mono | other |
| 1796 | Mar. 21, 2011 | 20.6 | 19.6 | 55.1 | 10.2 | 39.5 | 89.8 | 4.7 | 1.6 | 60.1 | 33.6 |
| 1797 | Mar. 22, 2011 | 56.5 | 51.3 | 72.0 | 34.5 | 29.1 | 65.5 | 24.4 | 7.6 | 36.0 | 31.9 |
| 1807 | May 17, 2011 | 52.1 | 68.0 | 78.9 | 24.3 | 49.3 | 75.7 | 9.3 | 0.5 | 44.8 | 45.4 |
| 1836 | Aug. 24, 2011 | 53.9 | 57.2 | 46.6 | 25.8 | 30.7 | 74.2 | 5.2 | 0.5 | 67.6 | 26.8 |
| 1884 | Apr. 18, 2012 | 71.6 | 85.1 | 43.1 | 20.1 | 32.9 | 79.9 | 30.8 | 9.7 | 18.4 | 41.1 |
| 1913 | Sep. 24, 2012 | 56.2 | 86.4 | 85.2 | 23.5 | 23.7 | 76.5 | 29.9 | ND | 27.9 | 24.9 |
| 1922* | Apr. 22, 2013 | 74.5 | 67.3 | 81.5 | 51.3 | 35.6 | 48.7 | 62.4 | ND | 14.7 | 12.8 |
| 1934* | Dec. 12, 2012 | 85.1 | 86.1 | 88.5 | 13.7 | 68.4 | 86.3 | 47.5 | ND | 20.3 | 21.5 |
| 1938* | Apr. 22, 2013 | 47.1 | 30.5 | 79.5 | 54.0 | 38.0 | 46.0 | 23.8 | ND | 61.5 | 6.7 |
| | average | 57.5 | 61.3 | 70.0 | 28.6 | 38.6 | 71.4 | 26.4 | 4.0 | 39.0 | 27.2 |
| | STDEV | 18.59 | 24.23 | 17.22 | 15.33 | 13.32 | 15.33 | 19.35 | 4.35 | 20.31 | 12.52 |
| | SEM | 6.20 | 8.08 | 5.74 | 5.11 | 4.44 | 5.11 | 6.45 | 1.45 | 6.77 | 4.17 |

Among viable tumor cells, a variety of cell surface or intracellular antigens were detected by flow cytometry. The expression of the antigens is shown in Table 5 below.

TABLE 5
Expression of tumor antigens in human ovarian tumor samples (%)

| Tumor | Mesothelin | Her-2 | IL-13Rα2 | Survivin | AIM2 | RANBP2 | gp100 | CD133 |
|---|---|---|---|---|---|---|---|---|
| 1796TuTcE | 4.72 | 72.30 | 35.60 | 79.00 | 0.13 | 95.00 | 0.45 | 4.07 |
| 1797TuTcE | 4.29 | 99.80 | 24.60 | 97.00 | 0.16 | 92.70 | 2.19 | 12.2 |
| 1807TuTcE | 6.53 | 89.90 | 34.00 | 99.30 | 0.28 | 90.20 | 1.87 | 12.2 |
| 1836 TuTcE | 61.50 | 93.90 | 34.30 | 93.90 | 0.06 | 76.10 | 0.48 | 13.5 |
| 1884 TuTcE | 4.50 | 42.00 | 18.60 | 68.40 | 0.61 | 46.70 | 1.98 | 0.12 |
| 1913 TuTcE | 28.30 | 85.60 | 35.60 | 90.20 | 0.32 | 59.90 | 0.87 | 1.95 |
| 1922 Bellco | 20.40 | 82.50 | 20.60 | 75.30 | 1.40 | 46.60 | 3.19 | 1.26 |
| 1934 TuTcE | 2.58 | 12.30 | 3.42 | 45.90 | 1.93 | 64.30 | 2.09 | 0.26 |
| 1938Bellco | 14.50 | 96.70 | 62.40 | 70.70 | 0.97 | 96.00 | 14.40 | 0.14 |
| average | 16.37 | 75.00 | 29.90 | 79.97 | 0.65 | 74.17 | 3.06 | 5.08 |
| SD | 17.98 | 27.61 | 15.28 | 16.21 | 0.62 | 19.24 | 4.10 | 5.48 |
| SEM | 5.99 | 9.20 | 5.09 | 5.40 | 0.21 | 6.41 | 1.37 | 1.83 |

Among all nine samples tested, high frequencies (>70%) of EpCAM⁺ cells had a HER2⁺, Survivin⁺ or RANBP2⁺ phenotype (Table 5). Lower but detectable levels of mesothelin and IL-13Rα2 were observed, although mesothelin expression was highly variable among specimens tested with some cells expressing no detectable levels of expression. GP100 levels when detectable were low. AIM2 was not detected in any primary ovarian cancer cell, but expressed at low levels in A375 control cells. CD133⁺ EpCAM+ cells (putative cancer stem cells) were detected at frequencies >1% in six of nine samples tested.

Table 6 provides the values for mean fluorescence intensity (MFI) in comparison to their matched isotype antibody control. Antigens that were expressed on the greatest frequency of EpCAM⁻ tumor cells, such as HER2, survivin, and RANBP2, were also expressed at the highest level, as shown by analysis of MFI.

TABLE 6
Expression of tumor antigens in human ovarian tumor samples (MFI)

| | Mesothelin | Her-2 | IL-13Rα2 | Survivin | AIM2 | RANBP2 | gp100 |
|---|---|---|---|---|---|---|---|
| Tumor | (Iso) | (Iso) | (Iso) | (Iso) | (Iso) | (Iso) | (Iso) |
| 1796TuTcE | 178 | 300 | 142 | 596 | 155 | 6327 | 229 |
| | (117) | (43.8) | (43.8) | (54.5) | (218) | (54.5) | (54.5) |
| 1797TuTcE | 99.6 | 5340 | 118 | 852 | 197 | 3852 | 339 |
| | (76.2) | (30.7) | (30.7) | (41.4) | (222) | (41.4) | (41.4) |
| 1807TuTcE | 104 | 467 | 187 | 2301 | 202 | 2672 | 339 |
| | (71.3) | (55.3) | (55.3) | (55.8) | (228) | (55.8) | (55.8) |
| 1836 TuTcE | 861 | 815 | 159 | 1113 | 137 | 1588 | 185 |
| | (149) | (50.5) | (50.5) | (48.9) | (201) | (48.9) | (48.9) |
| 1884 TuTcE | 159 | 156 | 99.2 | 330 | 96.5 | 810 | 141 |
| | (133) | (49) | (49) | (48.8) | (101) | (48.8) | (48.8) |
| 1913 TuTcE | 326 | 473 | 148 | 1038 | 168 | 1487 | 216 |
| | (150) | (44.6) | (44.6) | (57.3) | (179) | (57.3) | (57.3) |
| 1922 Bellco | 300 | 338 | 78.6 | 612 | 191 | 1198 | 278 |
| | (150) | (24.1) | (24.1) | (47.3) | (227) | (47.3) | (47.3) |
| 1934 TuTcE | 164 | 139 | 99.6 | 269 | 103 | 792 | 130 |
| | (129) | (58) | (58) | (52.6) | (115) | (52.6) | (52.6) |
| 1938Bellco | 151 | 687 | 207 | 2359 | 472 | 1.36E+04 | 654 |
| | (122) | (37) | (37) | (192) | (484) | (192) | (192) |
| average | 260.29 | 968.33 | 137.60 | 1052.22 | 191.28 | 3591.78 | 279.00 |
| | (121.28) | (43.67) | (43.67) | (66.51) | (219.44) | (66.51) | (66.51) |
| SD | 238.55 | 1654.63 | 42.76 | 778.47 | 112.10 | 4158.01 | 159.83 |
| | (29.27) | (11.25) | (11.25) | (47.31) | (110.14) | (47.31) | (47.31) |
| SEM | 79.52 | 551.54 | 14.25 | 259.49 | 37.37 | 1386.00 | 53.28 |
| | (9.76) | (3.75) | (3.75) | (15.77) | (36.71) | (15.77) | (15.77) |

Conclusions: The above results suggest an opportunity for immune-based therapy for advanced ovarian cancer. In particular, expression levels of HER2, survivin, and RANBP2 suggest that these molecules may allow for near universal therapy among ovarian cancer patients. Mesothelin and IL-13Rα2 also represent reasonable targets, though their expression level is moderate, and in some patients' cancer cells, completely lacking.

In sum, these data provide a rationale for the creation of immunotherapy targeting a broad array of antigens including HER2, mesothelin, IL-13Rα, survivin, and RANBP2 for women with ovarian cancer.

### Example 5

### Quantitative Real-Time PCR-Based Analysis of Gene Expression in Human Ovarian Cancer Cells, Cancer Stem Cells, and Ovarian Cancer Daughter Cells

Objective: To compare the gene expression of antigens in human ovarian cancer cells, cancer stem cells, and ovarian cancer daughter cells using real-time PCR (RT-PCR).

Materials & Methods:

1. Antigens: Her-2, IL-13Rα2, mesothelin, survivin, CD133, gp100, EGFR, AIM2

2. PCR TaqMang gene expression probes and reagents:
MSLN (Mesothelin) gene expression assay, Life Technologies, Part#Hs00245879_m1;
HER2 gene expression assay, Life Technologies, Part#Hs01001580_m1;
IL-13Rα2 gene expression assay, Life Technologies, Part#Hs00152924_m1;
BIRC5 (Survivin) gene expression assay, Life technologies, Part#Hs03043576_m1;
PROM1 (CD133) gene expression assay, Life Technologies, Part#Hs01009250_m1
PMEL (gp100) gene expression assay, Life Technologies, Part#Hs00173854_m1
AIM2 (Custom TaqMan® Gene Expression Assay), Life Technologies, Cat#4331348
GAPDH gene expression assay, Life Technologies, Part#Hs02758991_g1
EGFR gene expression assay, Life Technologies, Part#Hs01076078_m1
TaqMan gene expression master mix; Life technologies, part#4369016
Rneasy Mini Kit RNA isolation (cat#74104, Qiagen)
High Capacity cDNA Reverse Transcription Kit with RNase Inhibitor (cat#4374966, Life Technologies)

3. Cell Lines: human ovarian cancer cells (AC) 882AC and 1031AC, cancer stem cells (CSC) 882CSC and 1031 CSC, ovarian cancer daughter cells (ADC) 882 ADC and 1031 ADC

4. Human Ovarian Cancer Cells (AC) Culture

Ovarian cancer cell lines 882AC and 1031AC were cultured in McCoy's 5A medium (Mediatech, Herndon, Va.) supplied with 10% fetal bovine serum (Omega Scientific, Inc.) and Pen Strep Glutamine (100×) (Invitrogen). All cells were cultured in 5% CO₂ and at 37° C. in a cell incubator (Forma Scientific, Inc.).

5. Human Ovarian Cancer Stem Cells (CSC) Culture

Human ovarian cancers cells (882AC,1031AC) were grown in Dulbecco's modified Eagle's medium DMEM/F12 medium (Invitrogen) containing 10% fetal bovine serum (FBS) as growth medium and plated at a density of 1×10⁶ cells per 75 cm² cell culture flask (Corning Inc.). The cells attached and grew as a monolayer in flasks. The monolayers were then switched into DMEM/F12 medium supplemented with B-27 (Invitrogen, Carlsbad, Calif.), 20 ng/ml of basic fibroblast growth factor, and 20 ng/ml of endothelial-derived growth factor (Peprotech, Rocky Hill, N.J.).

6. Human Ovarian Cancer Daughter Cells (ADC) Culture

Human ovarian cancer stem cells (882CSC, 1031CSC) were grown in Dulbecco's modified Eagle's medium DMEM/F12 medium (Invitrogen) containing 10% fetal bovine serum (FBS) as growth medium and plated at a density of 1×10⁶ cells per 75 cm² cell culture flask (Corning Inc.). The cells attached and grew as a monolayer in flasks in about 2-3 weeks.

7. RNA extraction, cDNA synthesis, and qPCR

Total RNA was extracted from cell lines 882AC, 882CSC, 882ADC, and 1031AC, 1031CSC, and 1031ADC using Rneasy Mini Kit (Qiagen) according to the manufacturer's instructions. The complementary DNA was synthesized using High-Capacity® cDNA Reverse Transcription (cat#4374966), Life Technologies, CA) following the manufacturer's protocol.

The real-time PCR reactions were performed according to the manufacturer's instructions. The reaction consisted of 8.0 μl cDNA (42 ng), 10 μl TaqMan PCR Master Mix, 1.0 μl nuclease-free water and the following 1.0 μl TaqMan PCR probes (20×) for these genes: Hs01001580_m1 (HER2), Hs00152924_m1 (IL-13Ra2), Hs00245879_m1 (mesothelin), Part#Hs03043576_m1 (BIRC5, Survivin), Part#Hs01009250_m1 (PROM1,CD133), Part#Hs00173854_m1 (PMEL,GP100), Cat#4331348 (AIM2 Custom probe), Part#Hs01076078_m1 (EGFR), as well as internal control Hs02758991_g1 (GAPDH).

The reactions were performed on Bio-Rad iQ5 Real Time PCR system with the following thermal cycles: one cycle of 50° C. for 2 minutes and 95° C. for 10 minutes, followed by 40 cycles with a denaturation at 95° C. for 15 seconds and an annealing/extension at 56° C. for 60 seconds, extension at 72° C. for 30 seconds and a final extension step at 72° C. for 5 min. A melting curve was determined at the end of each reaction to verify the specificity of the PCR reaction. Ct Data analysis was performed using the Bio-Rad software supplied with the IQ5 Cycler system.

8. Data analysis using [2̂−{(ΔCt)] Method

Relative quantities for each antigen gene were calculated using the comparative [2̂−Δ(ΔCt)] method. The Ct value represents the cycle number at which the fluorescence passes the defined threshold. Delta Ct values (delta Ct=Ct_{test gene}−Ct_{mean of control genes}) were used to compare the difference of gene expression. Ct values of antigens gene expression levels were normalized to GAPDH and comparative Ct method [2̂−Δ(ΔCt)] was used to evaluate the gene expression.

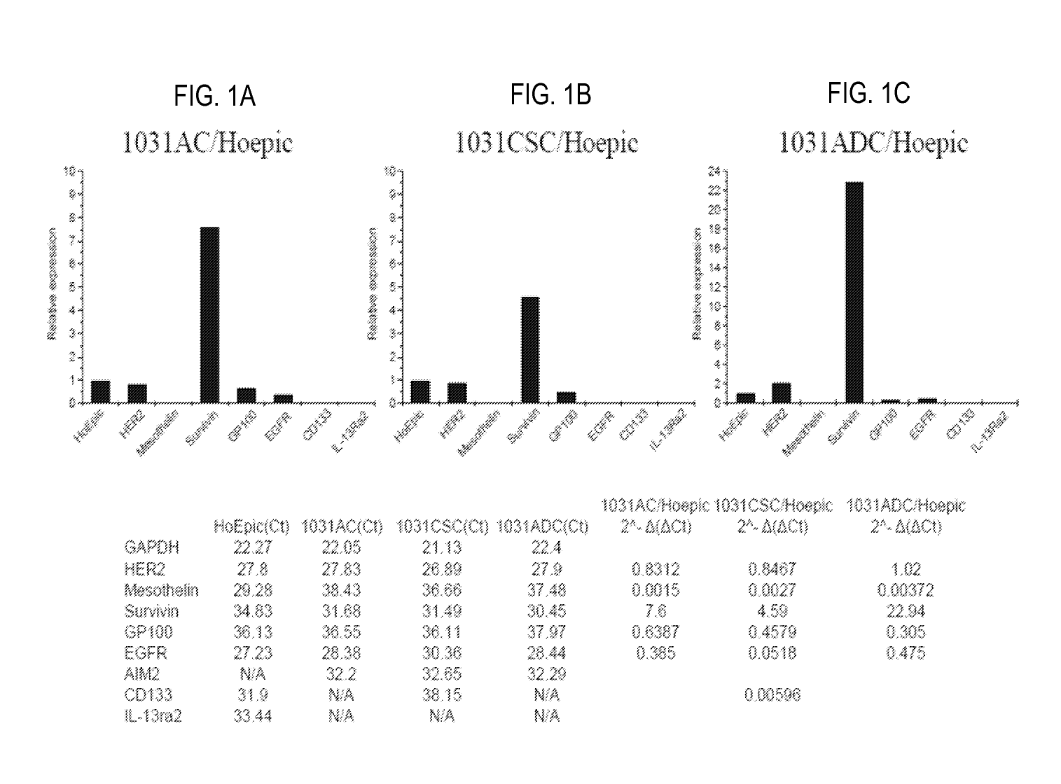

Results: The gene expression of HER2, mesothelin, survivin, gp100, EGFR, AIM2, CD133, IL-13Rα2 was evaluated in human ovarian cancer cells (1031AC), cancer stem cells (1031CSC), and ovarian cancer daughter cells (1031ADC). As shown in FIG. 1, the relative gene expression of HER2, mesothelin, survivin, gp100, and EGFR in 1031AC were 0.8312, 0.0015, 7.6, 0.637, and 0.385, respectively. These results suggest that the gene expression of survivin was higher (7.6 fold change) in 1031AC relative to control cell, whereas the gene expression of HER2, mesothelin, gp100 and EGFR in 1031AC was lower than that on the control cell. The relative gene expression of HER2, mesothelin, survivin, gp100, and EGFR in 1031CSC were 0.8467, 0.0027, 4.59, 0.4579, and 0.0518, respectively. These results suggest that the relative gene expression of survivin was higher (4.59 fold change) in 1031CSC relative to the control cell, whereas the relative gene expression of HER2, mesothelin, gp100, and EGFR in 1031CSC were lower expression than that in control cell. The relative gene expression of HER2, mesothelin, survivin, gp100, and EGFR in 1031ADC were 1.02, 0.00372, 22.94, 0.305, and 0.475, respectively. These results suggest that the relative gene expression of survivin was higher (22.94 fold) in 1031ADC relative to control cell, whereas the relative gene expression of HER2, mesothelin, gp100, and EGFR in 1031ADC was lower expression than that in control cell. The gene expression of AIM2 is only detectable in 1031AC, 1031CSC and 1031ADC, and is undetectable in the control cell. The gene expression of CD133 is only detectable in 1031CSC, and is undetectable in both 1031AC and 1031ADC. The gene expression of IL-13Rα2 is undetectable in 1031AC, 1031CSC and 1031ADC, and at low levels in the control cell.

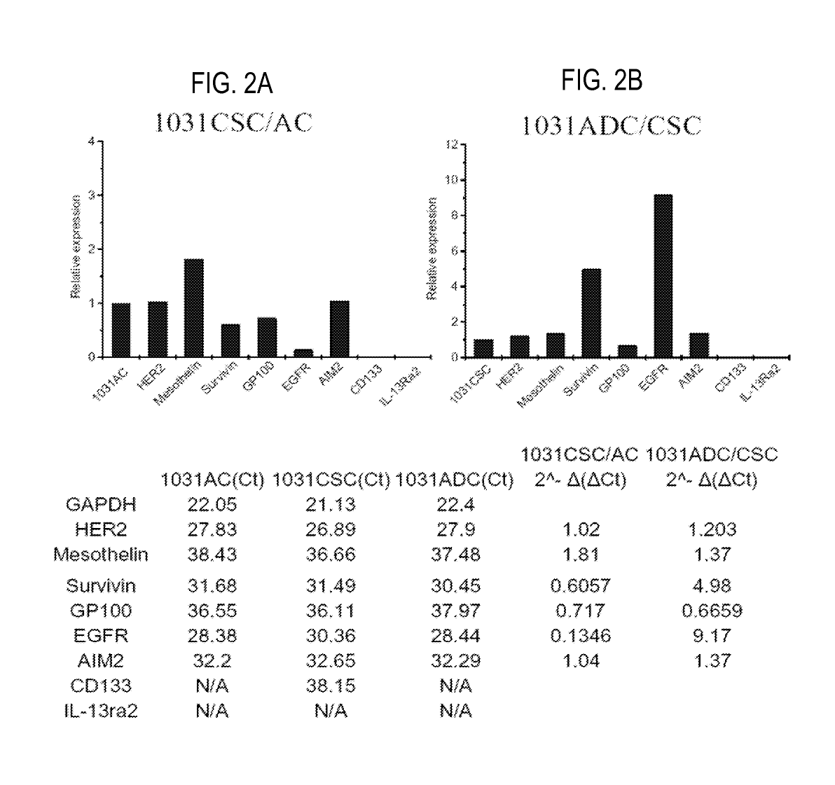

The gene expression of HER2, mesothelin, survivin, gp100, EGFR, AIM2, CD133, and IL-13Rα2 was compared amongst human ovarian cancer cells (1031AC), cancer stem cells (1031CSC), and ovarian cancer daughter cells (1031ADC). As shown in FIG. 2, the relative gene expression of HER2, mesothelin, survivin, gp100, EGFR, and AIM2 was 1.02, 1.81, 0.6057, 0.717, 0.1346, and 1.04 fold in 1031CSC relative to 1031AC. These results suggest that the gene expression of HER2, mesothelin and AIM2 in 1031CSC were a little higher than that in 1031AC,whereas the relative gene expression of survivin, gp100, and EGFR in 1031CSC were a little lower level than that in 1031AC. The relative gene expression of HER2, mesothelin, survivin, gp100, EGFR, AIM2, CD133, and IL-13Rα2 were 1.203, 1.37, 4.98, 0.6659, 9.37, 1.37 fold in 1031ADC relative to 1031CSC. These results suggest that the gene expression of HER2, mesothelin, and AIM2 in 1031ADC were a little higher than in 1031CSC, whereas survivin and EGFR were over-expressed in 1031ADC compared to 1031CSC. The gene expression of gp100 in 1031ADC was lower than that in 1031CSC. The gene expression of CD133 was only detectable in 1031CSC, and was undetectable either in 1031AC or 1031ADC. The gene expression of IL-13Rα2 was undetectable in 1031AC, 1031CSC, and 1031ADC, and at a lower level in control cells.

Conclusion:

1. Based on the Ct value of q-PCR, HER2, mesothelin, survivin, gp100, EGFR, and AIM2 were expressed in ovarian cancer cells (1031AC), ovarian cancer stem cells (1031CSC) and ovarian cancer daughter cells (1031ADC). The Ct of CD133 was only detectable in 1031CSC and was undetectable in 1031AC and 1031ADC under the experimental conditions. The Ct of IL-13Rα2 was undetectable in 1031AC, 1031CSC, and 1031ADC under the experimental conditions used herein.

2. The relative gene expression of survivin was 7.6, 4.59, and 22.94 fold in 1031AC, 1031CSC, and 1031ADC, respectively, suggesting that survivin has a higher level of expression in 1031AC, 1031CSC, and 1031ADC relative to control cells. The relative gene expression of HER2 in 1031ADC was 1.02 fold, suggesting that the expression in 1031ADC was a little higher than that in control cell, whereas the expression of HER2 in 1031AC and 1031CSC was lower level relative to control cell.

3. The gene expression of HER2, mesothelin, AIM2, and survivin in 1031CSC and 1031ADC were 1.02, 1.203; 1.81, 1.37; 1.04, 1.37; and 0.6057, 4.98 fold relative to 1031AC and 1031CSC, respectively, indicating that the genes expression of HER2, mesothelin, AIM2, and survivin in 1031ADC were higher than that in 1031CSC, and the gene expression of HER2, mesothelin, AIM2 in 1031CSC were higher than that in 1031AC. The gene expression of survivin in 1031CSC was lower level than that in 1031AC.

4. CD133 showed lower level expression in 1031CSC and undetectable expression in either 1031AC or 1031ADC. IL-13Rα2 was undetectable in 1031AC, 1031CSC, and 1031ADC under the experimental conditions used, suggesting that these genes are expressed at a lower level in these cells.

Taken together, identification of unique genes expression molecular signatures of HER2, mesothelin, survivin, gp100, EGFR, AIM2, CD133, and IL-13Rα2 provide a framework for the rational design of immunotherapy target for human ovarian cancer cell, cancer stem cell and ovarian cancer daughter cell.

### Example 6

### Analysis of the Expression of Tumor Antigens in Human Ovarian Tumor Cancer Cells, Cancer Stem Cells, and Ovarian Cancer Daughter Cells Based on Flow Cytometric Assay

Objective: To utilize flow cytometry-based analysis of antigen expression profiles in primary human ovarian cancer cells, cancer stem cells, and ovarian cancer daughter cells for potential immunotherapeutic targeting.

Materials & Methods:

### 1. Reagents

DMEM/F12: Invitrogen, Cat#11330-057 (Lot#1184632, Lot#1109388, Lot#891768);

McCoy's 5A, 1×: Mediatech, Inc, cat#10-050-CV (Lot#10050090, Lot#10050088);

B-27 supplement (50×): Invitrogen, cat#12587-010 (Lot#1192265, Lot#1153924, Lot#1079052);

Fetal Bovine Serum: Omega Scientific, Inc. Cat#FB-11 (Lot#170108, Lot#110300);

Pen Strep Glutamine: Invitrogen, cat#10378-016 (Lot#1030595);

Human FGF-basic: PeproTech, cat#100-18B (Lot#041208-1, Lot#051108);

Human EGF: cat#AF-100-15 (Lot#0212AFCO5, Lot#0711AFCO5, Lot#0211AFCO5-1, Lot#0911AFCO5-1);

BD Cytofix/cytoperm, Fixation and permeabilization kit. Cat#51-6896KC(Lot#81617); and

The antibodies used for the flow cytometric assay were as follows: PE-labeled antibodies against human survivin were from R&D Systems (Minneapolis, Minn.); PE-labeled antibodies against human HER-2/neu, IL-13Rα2, and EGFR were from Biolegend (San Diego, Calif.); PE-labeled antibody against human CD133 was from Miltenyi Biotec(San Diego, Calif.); antibody against human gp100 was from AbCam (Cambridge, Mass.); and antibody against human mesothelin was from Santa Cruz Biotechnology (Dallas, Tex.).

### 2. Cell Lines

Primary human ovarian cancer cells (AC): 882AC, 1031AC, 1078AC, 1082AC, 1077AC, 1105AC, and 1064AC;

Human ovarian cancer stem cells (CSC): 882CSC, 1031CSC, 1078CSC, and 1082CSC;

Human ovarian cancer daughter cells (ADC): 882ADC, 1031ADC, and 1078ADC; and

SKOV3 human ovarian cancer cell (American Type Culture Collection).

### 3. Human Ovarian Cancer Cells (AC) Culture

Human ovarian cancer cell lines (AC) (882AC, 1031AC, 1078AC, 1082AC, 1077AC, 1105AC, 1064AC, and SKOV3) were cultured in McCoy's 5A medium (Mediatech, Herndon, Va.) supplied with 10% fetal bovine serum (Omega Scientific, Inc.) and Pen Strep Glutamine (100×) (Invitrogen). All cells were cultured in 5% CO₂ and 37° C. in a cell incubator (Forma Scientific, Inc).

### 4. Human Ovarian Cancer Stem Cells (CSC) Culture

Human ovarian cancers cells (AC) (882AC, 1031AC, 1078AC, 1082AC) were grown in Dulbecco's modified Eagle's medium DMEM/F12 medium (Invitrogen) containing 10% fetal bovine serum (FBS) as growth medium and plated at a density of 1×10⁶ cells per 75 cm² cell culture flask (Corning Inc.). The cells attached and grew as a monolayer in flasks. Then, these monolayer cells were switched into DMEM/F12 medium supplemented with B-27 (Invitrogen, Carlsbad, Calif.), 20 ng/ml of basic fibroblast growth factor, and 20 ng/ml of endothelial-derived growth factor (Peprotech, Rocky Hill, N.J.).

### 5. Human Ovarian Cancer Daughter Cells (ADC) Culture

Human ovarian cancer stem cells (ADC) (882ADC,1031ADC,1078ADC) were grown in Dulbecco's modified Eagle's medium DMEM/F12 medium (Invitrogen) containing 10% fetal bovine serum (FBS) as growth medium and plated at a density of 1×10⁶ cells per 75 cm² cell culture flask (Corning Inc.). The cells attached and grew as a monolayer in flasks in about 2-3 weeks.

### 6. Flow Cytometric Analysis

The human ovarian cancer cells, cancer stem cells, and ovarian cancer daughter cells (0.5×10⁶ or 1×10⁶) were resuspended in 1% FBS-PBS and stained with the following specific PE labeled antibodies: PE-labeled antibodies against human survivin from R&D Systems (Minneapolis, Minn.); PE-labeled antibodies against human HER-2/neu, IL-13Rα2, and EGFR from Biolegend (San Diego, Calif.); PE-labeled antibody against human CD133 from Miltenyi Biotec (San Diego, Calif.); antibody against human gp100 from AbCam (Cambridge, Mass.); and antibody against human mesothelin from Santa Cruz Biotechnology (Dallas, Tex.).

For intracellular antigens (gp100) staining, cells were permeabilized using Cytofix/Cytoperm kit (BD Biosciences) and stained with PE-conjugated 2nd antibody.

Flow cytometric analysis was performed using a CyAn™ flow cytometer (Beckman Coulter) and the data was analyzed using Summit (Dako, Carpinteria, Calif.) software.

Results: In this study, expression of several antigens was tested using a FACS assay in seven primary human ovarian cancer cells, four human ovarian cancer stem cells, and three human ovarian cancer daughter cells. The expression results are listed in Tables 7-9.

TABLE 7
Expression of tumor antigens in human ovarian tumor samples (%)

| Tumor ID | Mesothelin | HER2 | IL13Rα2 | Survivin | CD133 | EGFR | gp100 |
|---|---|---|---|---|---|---|---|
| 882-CSC | 2.24 | 84.33 | 15.67 | 29.58 | 2.17 | 65.87 | 10.2 |
| 882-AC | 1.57 | 95.75 | 18.39 | 7.05 | 0.88 | 88.98 | 4.76 |
| 882-ADC | 2.27 | 97.78 | 5.4 | 8.19 | 4.02 | 92.82 | 0.62 |
| 1031-CSC | 2.12 | 49.93 | 9.67 | 25.3 | 1.45 | 46.24 | |
| 1031-AC | 1.36 | 97.57 | 5.03 | 6.78 | 1.42 | 93.98 | |
| 1031-ADC | 2.49 | 98.59 | 5.06 | 10.97 | 0.38 | 96.99 | |
| 1078-CSC | 2.58 | 83.94 | 31.47 | 36.43 | 10.77 | 17.78 | 0.2 |
| 1078-AC | 1.55 | 99.16 | 58.81 | 6.31 | 1.37 | 91.61 | 23.59 |
| 1078-ADC | 2.89 | 96.15 | 93.79 | 17.02 | 3.58 | 88.73 | |
| 1085AC | 1.65 | 86.87 | 31.55 | 11.79 | 3.12 | 59.37 | |
| Average | 2.072 | 89.007 | 27.484 | 15.942 | 2.916 | 74.237 | 7.874 |
| SD | 0.514 | 14.98 | 28.76 | 10.81 | 3.01 | 26.34 | 9.66 |
| SEM | 0.163 | 4.73 | 9.09 | 3.419 | 0.948 | 8.34 | 4.32 |

Table 7 is a summary of the expression of antigens of interest in four primary human ovarian cancer cells, three human ovarian cancer stem cells, and three human ovarian daughter cells. The results indicate that the average antigen expression of mesothelin, HER2, IL13Rα2, survivin, CD133, EGFR, and gp100 were 2.072%, 89.07%, 27.49%, 15.94%, 2.92%, 74.24% and 7.87%, respectively. The expression levels of mesothelin and CD133 were lower compared to the other antigens in ovarian cancer cells, cancer stem cells and ovarian cancer daughter cells. HER2 and EGFR were highly expressed in ovarian cancer cells, cancer stem cells, and ovarian cancer daughter cells.

Table 8 provides the values of mean fluorescence intensity (MFI) in comparison to their matched isotype antibody control (Iso). The MFI results indicated that the MFI of isotype Abs are lower than that of the MFI of antigen Abs.

TABLE 8
Expression of tumor antigens in human ovarian tumor samples (MFI)

| | | Meso | | HER2 | | IL13Rα2 | | Survivin | | CD133 |
|---|---|---|---|---|---|---|---|---|---|---|
| Tumor ID | Meso | (Iso) | HER2 | (Iso) | IL13Rα2 | (Iso) | Survivin | (Iso) | CD133 | (Iso) |
| 882-CSC | 10.6 | 6.48 | 21.86 | 6.48 | 19.13 | 6.48 | 23.67 | 6.48 | 15.57 | 6.48 |
| 882-AC | 8.33 | 10.28 | 22.69 | 10.28 | 18.9 | 10.28 | 34.13 | 10.28 | 16.59 | 10.28 |
| 882-ADC | 102.36 | 12.85 | 89.58 | 16.96 | 32.75 | 14.78 | 71.32 | 26.03 | 50.02 | 21.16 |
| 1031-CSC | 24.66 | 47.21 | 6.5 | 47.21 | 10.59 | 47.21 | 20.99 | 47.21 | 24.89 | 47.21 |
| 1031-AC | 42.83 | 7.35 | 48.02 | 6.75 | 16.3 | 6.75 | 39.52 | 6.63 | 35.26 | 9.96 |
| 1031-ADC | 28.75 | 2.86 | 29.52 | 3 | 10.85 | 4.41 | 8.36 | 2.87 | 18.55 | 2.16 |
| 1078-CSC | 17.73 | 29.82 | 81.21 | 29.82 | 95.99 | 29.82 | 43.53 | 29.82 | 86.23 | 29.52 |
| 1078-AC | 25.42 | 19.97 | 48.15 | 19.97 | 42.58 | 19.97 | 39.61 | 19.97 | 30.39 | 19.97 |
| 1078-ADC | 15.49 | 9.05 | 50.9 | 9.05 | 53.6 | 9.05 | 47.95 | 9.05 | 22.37 | 9.05 |
| Skov3 | 79.95 | 58.12 | 500 | 58.12 | 121.8 | 58.12 | 17.88 | 58.12 | 55.4 | 58.12 |
| Avg. | 35.61 | 20.40 | 89.84 | 20.76 | 42.25 | 20.69 | 34.70 | 21.65 | 35.53 | 21.39 |
| SD | 31.34 | 18.83 | 146.45 | 18.72 | 38.26 | 18.64 | 18.04 | 18.76 | 22.44 | 18.51 |
| SEM | 9.91 | 5.95 | 46.17 | 5.91 | 12 | 5.89 | 5.7 | 5.93 | 7.09 | 5.85 |

As shown in Table 9, the HER2 and IL13Rα2 antigens were highly expressed in 1082AC, 1082CSC, 1077AC, 1105AC, and 1064AC, their expression levels being 82.03% and 44.97%, respectively. Mesothelin, CD133, and gp100 were expressed at lower levels. HER2 was also expressed at a high level in the SKOV3 human ovarian cancer cell. IL-13Rα2 and mesothelin were also expressed at a high level in A375 and Hela-229 cells, their expression levels being 82.87% and 55.9%, respectively.

TABLE 9
Expression of tumor antigens in human ovarian tumor samples (%)

| Tumor ID | Meso | HER2 | IL13Rα2 | Survivin | CD133 | EGFR | gp100 |
|---|---|---|---|---|---|---|---|
| 1082-CSC | 3.96 | 87.64 | 53.15 | 45.8 | 7.62 | 0.6 | |
| 1082-AC | 5.22 | 98.25 | 7.25 | 9.25 | 2.68 | 1.65 | 7.8 |
| 1077-AC | 1.55 | 83.63 | 88.73 | 7.84 | 0.72 | 37.55 | |
| 1105-AC | 3.28 | 78.59 | 4.5 | 80.73 | 7.89 | | |
| 1064-AC | 1.27 | 62.05 | 71.22 | 4.61 | 0.12 | 75.87 | |
| Avg. | 3.06 | 82.03 | 44.97 | 29.65 | 3.81 | 28.92 | |
| SD | 1.66 | 13.31 | 37.85 | 33.12 | 3.73 | 35.7 | |
| SEM | 0.74 | 5.94 | 16.92 | 14.82 | 1.66 | 17.85 | |
| SKOV3 | 1.23 | 99.5 | 0.51 | 1.91 | 0.63 | 1.55 | 14.07 |
| Avg. | 1.23 | 99.5 | 0.51 | 1.91 | 0.63 | 1.55 | |
| SD | 0.94 | 0.63 | 0.19 | | 0.15 | | |
| SEM | 0.54 | 0.36 | 0.11 | | 0.11 | | |
| A375 | | | 82.87 | | | | |
| Hela-229 | 55.9 | | | | | | |

Conclusion: The above results demonstrated that ovarian cancer cells, ovarian cancer stem cells, and ovarian cancer daughter cell express the tested antigens. Of these, HER2 and EGFR showed the highest expression, and the expression of IL13Rα2, survivin was at a moderate level. gp100, mesothelin, and CD133 were expressed at a lower level; however, in view of their RNA expression levels based on the q-PCR assay, they are still good candidates for immunotherapy targets.

Taken together, a vaccine based on the antigens of HER2, mesothelin, survivin, gp100, EGFR, AIM2, CD133, and IL-13Rα2 can target human ovarian cancer cells, cancer stem cells, as well as ovarian cancer daughter cells. Moreover, the antigens up-regulated expressions in ovarian cancer stem cells compared to ovarian cancer cells and daughter cells based on FACS data in Table 7 provide a new target cell for immunotherapy specifically targeting ovarian cancer stem cells.

### Example 7

### IFN-γ ELISPOT Assay of Antigen-Specific T Cell Response

Objective: To conduct an IFN-γ ELISPOT assay to check the the antigen-specific T cell response to the CD133 HLA-A2 peptides: CD133p405, CD133p753, and CD133p804.

In order to develop new generation of immunotherapy targets for ovarian cancer cell and ovarian cancer stem cells, we proposed the above HLA-A2 peptides as potential targets. We hypothesized that CD133 HLA-A2 A2 peptides could induce an antigen-specific immune response.

To test this hypothesis, effector CD8^{+′} T cells were isolated and co-cultured with HLA-A2+ DC pulsed with CD133 peptides to induce antigen-specific CTLs. Antigens-specific T cell responses were evaluated by an IFN-γ ELISPOT assay.

### Materials & Methods:

### Generation of Human Dendritic Cells

Human monocyte-derived DC was generated using previously described methods. Briefly, monocytes were isolated from PBMC by magnetic immunoselection using EasySep human monocyte enrichment kit (Stem Cell Technologies) in accordance with the manufacturer's instructions and then cultured at 5×10⁷/ml in 20 ml of GMP CellGenix DC serum-free medium (Cat#20801-0500, Cellgenix) supplemented with 1000 unit/ml of recombinant human GM-CSF (Cat#AF-300-03, Peprotech, Inc) and recombinant human IL-4(Cat#AF-200-04, Peprotech, Inc). Cells were harvested after 3 or 6 days of culture. The DCs were washed and plated in 6-well plates at a concentration of 5×10⁶ cells/well IFN-γ (1000 unit/ml) and monophosphoryl lipid A (MPLA, 20-50 μg/ml) was added into the wells to mature the DC for 24 hr or 48 hrs. Prior to some assays, DC was frozen and stored into liquid nitrogen.

### CTL-Induction and Detection of Mart1-Specific CD8⁺ by HLA-A*0201/Mart1 Tetramers

In order to evaluate antigen-specific immune responses, CD8⁺ T cells were isolated from fresh or frozen apheresis by positive selection using Dynabeads® CD8 Positive Isolation Kit (Life Technologies, Grand Island, N.Y.) and co-cultured with autologous mDC for four weeks. DCs were added weekly. Briefly, mDC was pulsed with synthetic peptides (10 μg/μl) for 6-8 hours at 37° C., and then treated with 20 μg/ml Mitomycin C (Sigma-Aldrich, St. Louis, Mo.) for 25 min at 37° C. and 5% CO2. The mDCs (5×10⁴ cells/well) were co-cultured with autologous CD8⁺ T cells (5×10⁵ cells/ well) in a 96-well plate at 37° C., 5% CO₂ in a final volume of 200 μl CTL medium(IMDM with 0.24 mM Asparagine, 0.55 mM L-Arginine, 1.5 mM L-Glutamine and 10% heat inactivated human AB serum). Half of the medium was replaced every other day by fresh culture medium containing 40 IU/ml IL-2 and 20 ng/ml IL-7, and in the 3rd and 4th week 40 IU/ml of IL-2 was replaced with 25 ng/ml of IL-15. Peptides also could be added to the culture well at a final concentration of 1-2 μg/ml.

### IFN-γ ELISPOT Assay

Antigen-specific immune responses were evaluated by the IFN-γ Elispot kit (BD Biosciences) following previously described methods. Briefly, 1×10⁵ CTL cells were co-cultured with 7.5×10⁴ T2 cells pulsed with or without 10 μg/ml of peptides and seeded into 96-well plates for 20 hours. CTL cells without T2 cells and CTL plus 5 μg/ml PHA were set as negative and positive controls, respectively. The colored spots, representing cytokine-producing cells, were counted under a dissecting microscope. The results were evaluated by an automated ELISPOT reader system using KS ELISPOT 4.3 software.

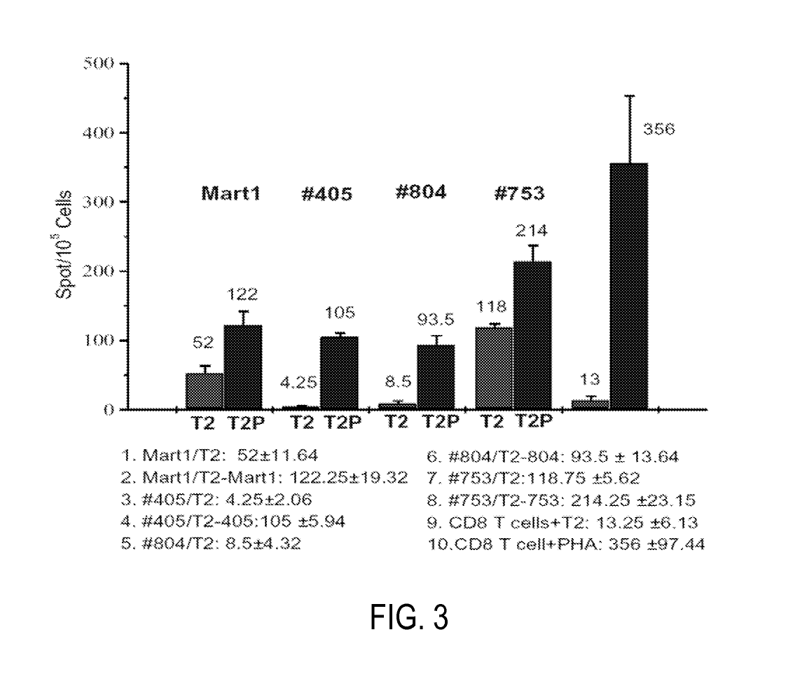

Results: As shown in FIG. 3, CTLs produce more IFN-γ against T2 cell loaded with the peptides compared with T2 control (no peptides). The results of IFN-γ ELISPOT assay indicated that CD133 peptides of CD133p405, CD133p753, and CD133p804-specific CTLs can efficiently recognize T2 pulsed with these antigens and boost the T cell immune response.

Conclusion: The IFN-γ ELISPOT assay demonstrated that CD133 peptides of CD133p405, CD133p753, and CD133p804-specific CTLs can efficiently recognize these antigens containing epitopes and induce T2 cell immune response. This result forms the basis to further develop immunotherapy target for human ovarian cancer cells and ovarian cancer stem cells as well as ovarian cancer daughter cells.

### Example 8

### Microarray Dataset Analyses Genes Expression Profiles and the Correlation Between RNA Expression and Overall Survival (OS)

Objective: To compare gene expression of genes of interest in human ovarian cancer and normal tissue from the TCGA microarray dataset and to determine whether the gene expression is associated with poor overall survival (OS) in patients with high-grade serous ovarian cancer.

Background: The goal of gene expression profiling studies is to identify gene expression signatures between tumor and normal tissue and to identify the correlation between gene expression and clinical outcome such as overall survival (OS) in order to discover potential biomarkers for treatment (e.g., for use as an immunotherapy target).

Methods: The Cancer Genome Atlas (TCGA) project has analyzed mRNA expression, microRNA expression, promoter methylation, and DNA copy number in 586 high-grade serous ovarian cystadenocarcinoma that were profiled on the Affymetrix U133A platform and preprocessed with dChip (version Dec. 5, 2011) software as described in the manual (Nature, 2011:609; Proc Natl Acad Sci USA 2001; 9:31).

GSE9891 contains the expression data and clinical data of 285 ovarian cancer samples and has been deposited in the Gene Expression Omnibus (GEO) (GSE9891) (Clin Cancer Res 2008; 14:5198).

The microarray dataset was analyzed for the RNA expression of genes of interest in human ovarian cancer samples. In addition, this example compared the correlation between RNA expression and overall survival (OS) of ovarian cancer patients.

Gene expression analysis tools at tcga-data.nci.nih.gov/tcga/, cancergenome.nih.gov, and oncomine.org were used to examine the RNA expression of ICT140 genes in 586 human serous ovarian cancer samples in TCGA dataset.

The Kaplan-Meier method was used to estimate the correlation between RNA expression and overall survival (OS) and the log-rank test was employed to compare OS across group. All analyses were performed using the web-based Kaplan-Meier plotter tool (kmplot.com). The overall survival curves and the number-at-risk were indicated below the main plot. Hazard ratio (HR; and 95% confidence intervals) and log-rank P values were also calculated.

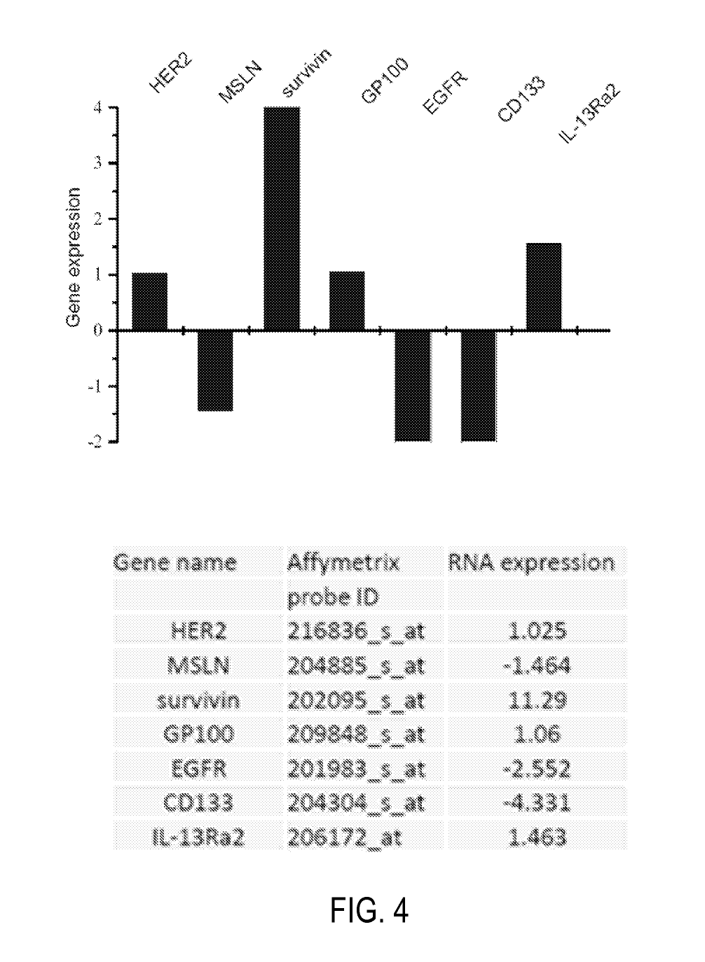

Results: As shown in FIG. 4, the mRNA expression value of HER2, survivin, gp100, and IL-13Rα2 were 1.025, 11.29, 1.06, and 1.463, respectively, in the TCGA ovarian cancer microarray dataset, indicating that the expression of these genes in ovarian cancer tissue were higher than that in normal tissue. In contrast, the expression value of mesothelin (MSLN), EGFR, and CD133 were −1.464, −2.552, and −4.331 indicating that the expression of these genes in ovarian cancer tissue is lower than that in normal tissue.

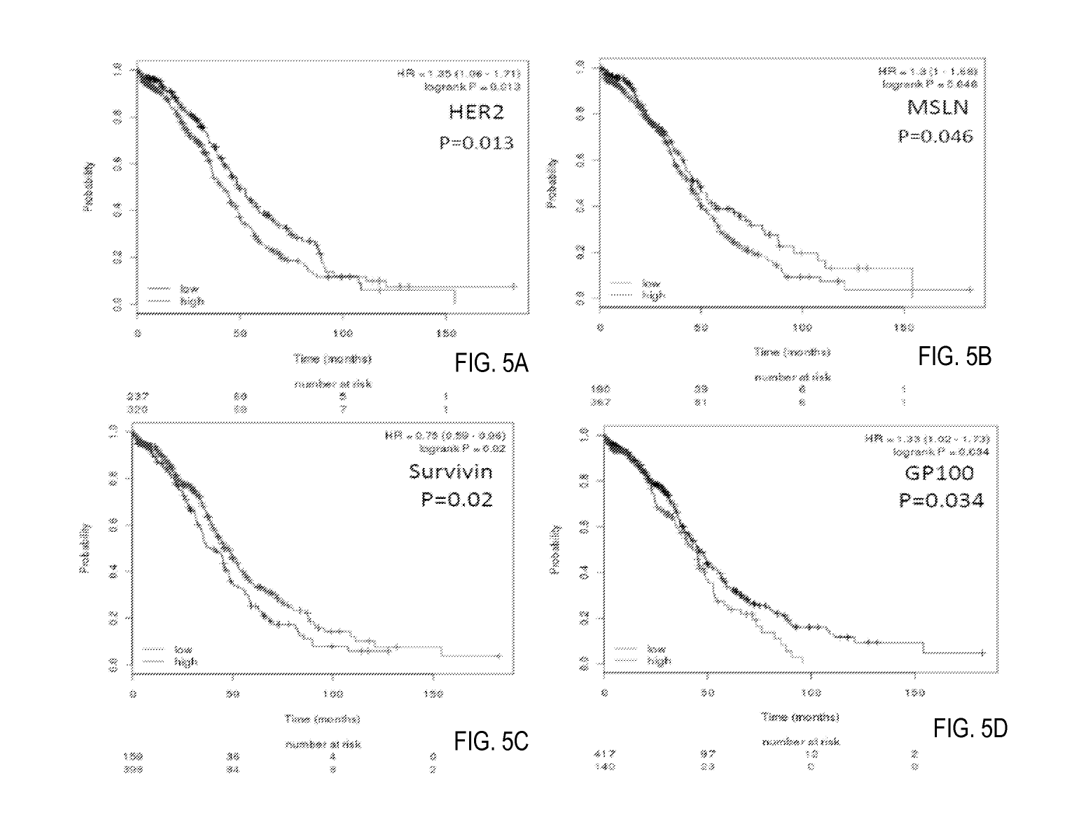

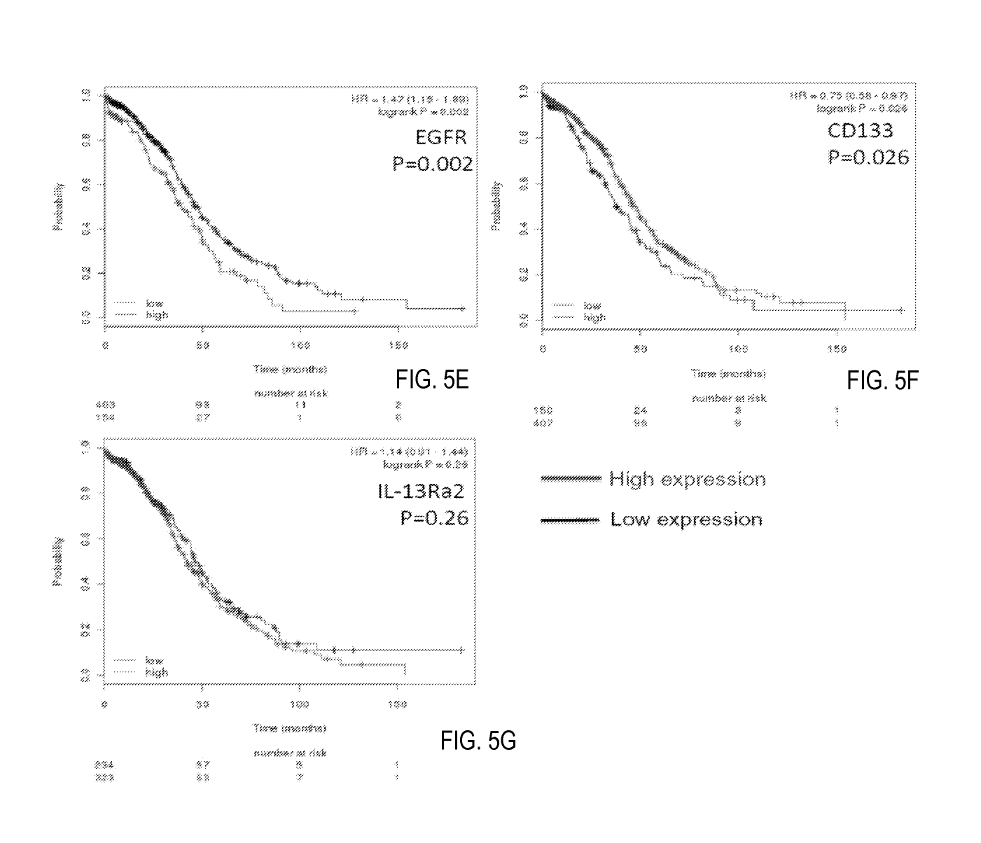

Correlation between RNA expression of these genes and the overall survival (OS) in ovarian cancer patients in TCGA microarray dataset was evaluated by comparing survival in patient groups with “high” and ‘low” RNA expression of these genes. For the TCGA dataset, the Kaplan-Meier results of overall survival (OS) for the patients in the “high” and “low” expression groups are depicted in FIG. 5A and FIG. 5B. The results in FIG. 5A and FIG. 5B showed that the patient group with “high” RNA expression of the genes HER2, MSLN, survivin, gp100, EGFR, and CD133 had poor overall survival (OS) with statistical significance (p<0.05), whereas there were no significant differences between overall survival (OS) and the RNA expression of IL-13Rα2 gene.

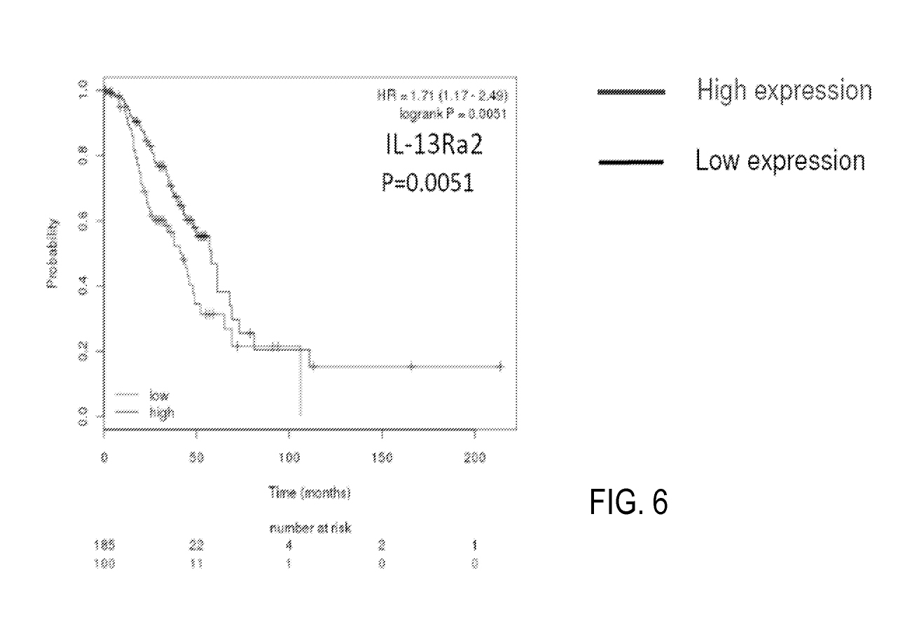

In order to validate the correlation between overall survival (OS) and RNA expression of IL-13Rα2, the GSE9891 dataset was analyzed and it was found that the patient group with “high” RNA expression of IL-13Rα2 had poor overall survival (OS) (FIG. 6).

Conclusion: These findings demonstrate that the proposed genes of HER2, MSLN, survivin, gp100, EGFR, CD133, and IL-13Rα2 are associated with poor overall survival (OS) in patients with high-grade ovarian cancer based on the TCGA and GSE9891 datasets. These results provide the basis for the rational design of novel treatment strategies including immunotherapy.

### Other Embodiments

It is to be understood that while the invention has been described in conjunction with the detailed description thereof, the foregoing description is intended to illustrate and not limit the scope of the disclosure. Other aspects, advantages, and modifications are within the scope of the following claims.

1. A pharmaceutical composition comprising a pharmaceutically acceptable carrier, an adjuvant, and a mixture of at least one major histocompatibility complex (MHC) class I epitope of at least five antigens selected from the group consisting of mesothelin, HER-2/neu, IL-13 receptor α2, survivin, CD133, gp100, AIM-2, and epidermal growth factor receptor (EGFR).

2.-5. (canceled)

6. The composition of claim 1, wherein the at least one MHC class I epitope is an HLA-A2 epitope.

7. The composition of claim 1, wherein the at least one MHC class I epitope is synthetic.

8. The composition of claim 1, further comprising at least one MHC class II epitope.

9.-10. (canceled)

11. A composition comprising isolated dendritic cells, wherein the dendritic cells present peptide sequences on their cell surface, wherein the peptide sequences comprise at least one major histocompatibility complex (MHC) class I epitope of at least five antigens selected from the group consisting of mesothelin, HER-2/neu, IL-13 receptor α2, survivin, CD133, gp100, AIM-2, and epidermal growth factor receptor (EGFR).

12.-14. (canceled)

15. The composition of claim 11, wherein the dendritic cells present peptide sequences comprising MHC class I epitopes of eight of the antigens.

16. The composition of claim 11, wherein the at least one MHC class I epitope is an HLA-A2 epitope.

17. The composition of claim 11, wherein the at least one MHC class I epitope is synthetic.

18. The composition of claim 11, wherein the dendritic cells further present at least one MHC class II epitope.

19. The composition of claim 11, further comprising an adjuvant.

20. The composition of claim 11, further comprising a pharmaceutically acceptable carrier.

21. The composition of claim 11, wherein the dendritic cells acquired the epitopes in vitro by exposure to synthetic peptides comprising the epitopes.

22. A method of treating an ovarian cancer, comprising administering to a subject in need thereof an effective amount of a composition of claim 11.

23. (canceled)

24. A method of killing ovarian cancer stem cells, comprising administering to a subject in need thereof an effective amount of a composition of claim 11.

25. (canceled)

26. The method of claim 22, further comprising administering a chemotherapeutic agent prior to, at substantially the same time as, or subsequent to, administering the subject with the composition.

27. The method of claim 26, wherein the chemotherapeutic agent is cyclophosphamide.

28. A process comprising:
obtaining bone marrow derived mononuclear cells from a patient;
culturing the mononuclear cells in vitro under conditions in which mononuclear cells become adherent to a culture vessel;
selecting adherent mononuclear cells;
culturing the adherent mononuclear cells in the presence of one or more cytokines under conditions in which the cells differentiate into antigen presenting cells;
culturing the antigen presenting cells in the presence of peptides, wherein the peptides comprise amino acid sequences corresponding to at least one MHC class I peptide epitope of at least five of the following antigens: mesothelin, HER-2/neu, IL-13 receptor α2, survivin, CD133, gp100, AIM-2, and EGFR, under conditions in which the antigen presenting cells present the peptides on major histocompatibility class I molecules.

29. The process of claims 28, wherein the one or more cytokines comprise granulocyte macrophage colony stimulating factor and interleukin-4 (IL-4).

30. The process of claim 28, wherein the one or more cytokines comprise tumor necrosis factor-α (TNF-α).

31. The process of claim 28, wherein the bone marrow derived cells are obtained from a patient diagnosed with epithelial ovarian cancer.
